(19) United States
(12) Patent Application Publication
(10) Pub. No.: US 2015/0245839 A1
(43) Pub. Date: Sep. 3, 2015

(54) URETHRAL ANASTOMOSIS DEVICE AND METHOD

(71) Applicant: ENDO PHARMACEUTICALS INC., Malvern, PA (US)

(72) Inventors: Anthony J. Wirtel, III, Malvern, PA (US); Rahul S. Nair, Thiruvananthapuram (IN); Cinish P. Varghese, Mannamkandam P.O. (IN); Rajesh Tulsiram Shelke, Horamavu Bangalore (IN); Sandeep Ambardekar, Dadar West (IN); Siddhi Shivkumar, Mulund West (IN); Mangesh Patankar, Navi Mumbai (IN); Nikhil Ramchandra Katre, Thane Distr. (IN); Dinesh A. Diwakar, Mumbai (IN); Jithin Ambujan, Nashik Maharashtra (IN); Rajkumar V. Shelke, Dist. Latur Maharashtra (IN); Divya Chetan Shenoy, Maharashtra (IN); Athar Anwar Solkar, Maharashtra (IN); Arvind Kumar Gupta, Uttar Pradesh (IN); Mohammad Afroz Alam, Bihar (IN); Debasish Pradhan, Sambalpur (IN); Salman Kapadia, Madhya Pradesh (IN)

(21) Appl. No.: 14/429,232

(22) PCT Filed: Sep. 17, 2013
(86) PCT No.: PCT/US13/60150

§ 371 Date: Mar. 18, 2015

Related U.S. Application Data
(60) Provisional application No. 61/702,645, filed on Sep. 18, 2012.

(51) Int. Cl. A61B 17/11

(52) U.S. Cl. CPC A61B 17/11

(57) ABSTRACT

Provided herein is a two-part coupling assembly for re-connecting a first hollow body part to a second body part and an instrument (90) and method for emplacement. The coupling assembly comprises coupling parts (2, 52) having securement elements (20, 70) that are actuated by separate deployment mechanisms of the instrument and attach to the first and second body parts. The first and second coupling parts having interconnecting elements (48, 62) that couple the two-part assembly together and re-connect the first and second body parts.

### CROSS REFERENCE TO RELATED APPLICATIONS

This application claims to the benefit of U.S. Provisional Patent Application No. 61/702,645, filed Sep. 18, 2012, the entire contents of which are incorporated by reference, as if fully set forth herein.

### TECHNICAL FIELD

This disclosure relates generally to the field of medical devices and, in particular, to devices and methods for reconnecting two hollow body parts, such as a urethra to a bladder.

### BACKGROUND

The prostate gland is a semen-producing organ located in the abdomen of males. Cancer of the prostate gland is an extremely common ailment among older American men. In fact, prostate cancer is the second-leading cause of cancer-related deaths and the most common cancer diagnosed in men. In 2010, an estimated 90,000 American men underwent radical prostatectomy, a surgery in which their prostate gland was removed. If past experience holds, nearly one-third of these men suffered complications, which at the least were painful and at most required further invasive surgery.

The most common complication, known as bladder-neck contracture, is caused by leakage of urine into the abdomen. During a radical prostatectomy, after the prostate is removed, it is necessary to re-attach the bladder (where the body stores urine) to the urethra (the passage carrying urine from the bladder to the penis). Unfortunately, the conventional hand-sewn five- to six-suture re-attachment (an anastomosis) often does not result in a leak-proof seal. Consequently, urine can leak from the bladder into the abdomen until the anastomosis is sealed, which can take up to five days. Such leakage causes scarring, which in turn leads to bladder-neck contractures. A patient suffering from such a contracture typically is unable to urinate and requires painful and expensive intervention.

In addition, with the robotic approach, the urethrovesicle anastomosis can be one of the most challenging components of the surgery. In the most-experienced hands, this can add thirty minutes to the operation, and in the hands of a novice, it can add one hour to the operation.

Accordingly, it can be seen that a need exists for improved ways to attach hollow body vessels, such as the urethra to the bladder. It is to this and other solutions that the embodiments of the present invention are primarily directed.

### BRIEF DESCRIPTION OF THE DRAWINGS

FIG. 1 is a perspective view of a first exemplary embodiment of a first ring assembly structure of an anastomosis device.

FIG. 2 is a further perspective view of the first ring assembly of FIG. 1.

FIG. 3 is a further perspective view of the first ring assembly of FIG. 1.

FIG. 4 is a cross-sectional view of the first ring assembly of FIG. 1, depicted in the retracted position.

FIG. 5 is a cross-sectional view of the first ring assembly of FIG. 1, depicted in the deployed position.

FIG. 6 is a perspective view of a first exemplary embodiment of a second ring assembly structure of an anastomosis device.

FIG. 6A is a perspective view of an alternative embodiment of a portion of the second ring assembly depicted in FIG. 6.

FIG. 6B is a perspective view showing alternative embodiments of a first ring assembly and a second ring assembly.

FIG. 6C is a partial perspective view of an exemplary embodiment showing a first ring assembly coupled to a second ring assembly.

FIG. 7 is a perspective view of a first exemplary embodiment of an anastomosis system.

FIG. 8 is an exploded view of the anastomosis system of FIG. 7.

FIG. 9A is a perspective view of a first exemplary embodiment of an actuation shaft used within an anastomosis device.

FIG. 9B is a further perspective view of the actuation shaft of FIG. 9A.

FIG. 10A is a further perspective view of the actuation shaft of FIGS. 9A and 9B, depicted with an adapter and rotary actuation knob.

FIG. 10B is a further perspective view of the actuation shaft of FIG. 10A.

FIG. 10C is a further perspective view of the actuation shaft of FIG. 10A.

FIG. 11 is a perspective view of the actuation shaft of FIGS. 9A and 9B, depicted with an adapter and rotary selection knob.

FIG. 12A is a perspective view of a first exemplary embodiment of a partially assembled exemplary handle assembly for an anastomosis device.

FIG. 12B is a further perspective view of the handle assembly of FIG. 12A.

FIG. 13A is a perspective view of a first exemplary embodiment of an implant support.

FIG. 13B is a further perspective view of the implant support of FIG. 13A.

FIG. 13C is a cross-sectional view of the implant support shown in FIGS. 13A and 13B.

FIG. 14A is a perspective view of the actuation shaft shown in FIGS. 10A-10C, depicted during a first stage of a deployment operation.

FIGS. 14B is a perspective view of the actuation shaft shown in FIG. 14A, depicted during a second stage of a deployment operation.

FIGS. 14C is a perspective view of the actuation shaft shown in FIG. 14A, depicted during a third stage of a deployment operation.

FIGS. 14D is a perspective view of the actuation shaft shown in FIG. 14A, depicted during a fourth stage of a deployment operation.

FIG. 14E is a perspective view of the actuation shaft shown in FIG. 14A, depicted during a fifth stage of a deployment operation.

FIG. 15A is a cross-sectional view of the handle assembly depicted in FIGS. 12A and 12B.

FIG. 15B is a further cross-sectional view of the handle assembly depicted in FIGS. 12A and 12B.

FIG. 16 is a further perspective view of the anastomosis system depicted in FIG. 7.

FIG. 17A is a cross-sectional view of a distal end of the anastomosis system depicted in FIG. 16.

FIG. 18A is a further cross-sectional view of the distal end of the anastomosis system depicted in FIG. 17A.

FIG. 18B is a further cross-sectional view of the proximal end of the anastomosis system depicted in FIG. 17B.

FIG. 19 is a perspective view of a second exemplary embodiment of an anastomosis system.

FIG. 20 is a perspective view of a third exemplary embodiment of an anastomosis system.

FIG. 21 is a perspective view of a shaft flexing portion of the anastomosis system of FIG. 20.

FIG. 22 is a perspective view of a fourth exemplary embodiment of an anastomosis system.

FIG. 23A is a perspective view of a second exemplary embodiment handle assembly for use with an anastomosis system.

FIG. 23B is a side view of the handle assembly shown in FIG. 23A.

FIG. 24A is a perspective view of a third exemplary embodiment handle assembly for use with an anastomosis system.

FIG. 24B is a side view of the handle assembly shown in FIG. 24A.

FIG. 25 is a further perspective view of the anastomosis system of FIG. 7, depicted during insertion into a patient.

FIG. 26A is a further perspective view of the anastomosis system depicted in FIG. 25, during a first stage of the insertion and deployment process.

FIG. 26B is a cross-sectional view of the anastomosis system shown in FIG. 26A.

FIG. 26C is a cross-sectional view of a handle portion of the anastomosis system of FIG. 26A.

FIG. 26D is a cross-sectional view of a distal portion of the anastomosis system of FIG. 26A.

FIG. 27A is a further perspective view of the anastomosis system depicted in FIG. 25, during a second stage of the insertion and deployment process.

FIG. 27B is a cross-sectional view of the anastomosis system shown in FIG. 27A.

FIG. 27C is a cross-sectional view of a handle portion of the anastomosis system of FIG. 27A.

FIG. 27D is a cross-sectional view of a distal portion of the anastomosis system of FIG. 27A.

FIG. 28A is a further perspective view of the anastomosis system depicted in FIG. 25, during a third stage of the insertion and deployment process.

FIG. 28B is a cross-sectional view of the anastomosis system shown in FIG. 28A.

FIG. 28C is a cross-sectional view of a handle portion of the anastomosis system of FIG. 28A.

FIG. 28D is a cross-sectional view of a distal portion of the anastomosis system of FIG. 28A.

FIG. 29A is a further perspective view of the anastomosis system depicted in FIG. 25, during a fourth stage of the insertion and deployment process.

FIG. 29B is a cross-sectional view of the anastomosis system shown in FIG. 28A.

FIG. 29C is a cross-sectional view of a handle portion of the anastomosis system of FIG. 29A.

FIG. 29D is a cross-sectional view of a distal portion of the anastomosis system of FIG. 29A.

FIG. 30A is a further perspective view of the anastomosis system depicted in FIG. 25, during a fifth stage of the insertion and deployment process.

FIG. 30B is a cross-sectional view of the anastomosis system shown in FIG. 30A.

FIG. 30C is a cross-sectional view of a handle portion of the anastomosis system of FIG. 30A.

FIG. 30D is a cross-sectional view of a distal portion of the anastomosis system of FIG. 30A.

FIG. 31A is a further perspective view of the anastomosis system depicted in FIG. 25, during a sixth stage of the insertion and deployment process.

FIG. 31B is a cross-sectional view of the anastomosis system shown in FIG. 31A.

FIG. 31C is a cross-sectional view of a handle portion of the anastomosis system of FIG. 31A.

FIG. 31D is a cross-sectional view of a distal portion of the anastomosis system of FIG. 31A.

FIG. 32A is a further perspective view of the anastomosis system depicted in FIG. 25, during a seventh stage of the insertion and deployment process.

FIG. 32B is a cross-sectional view of the anastomosis system shown in FIG. 32A.

FIG. 32C is a cross-sectional view of a handle portion of the anastomosis system of FIG. 32A.

FIG. 32D is a cross-sectional view of a distal portion of the anastomosis system of FIG. 32A.

FIG. 33A is a side view of a portion of a further alternative exemplary embodiment of a central ring in a retracted or undeployed position.

FIG. 33B is a side view of a portion of the central ring depicted in FIG. 33A in an extended or deployed position.

FIG. 34 is a perspective view of a further alternative embodiment of a first ring assembly in the undeployed position.

FIG. 35A is a side view of an alternative embodiment of a first ring securement element.

FIG. 35B is a side view of an alternative embodiment of a first ring securement element.

FIG. 35C is a side view of a further alternative embodiment of a first ring securement element.

FIG. 35D is a side view of an alternative embodiment of a first ring securement element.

FIG. 36A is a cross-sectional view of an alternative embodiment of a first ring assembly in an undeployed position.

FIG. 36B is a cross-sectional view of the alternative embodiment of a first ring assembly depicted in FIG. 36A in a partially deployed position.

FIG. 36C is a cross-sectional view of the alternative embodiment of a first ring assembly depicted in FIG. 36A in a fully deployed position.

FIG. 37A is a cross-sectional view of a further alternative embodiment of a distal portion of an anastomosis system.

FIG. 37B is a cross-sectional view of the distal portion of an anastomosis system depicted in FIG. 37A, after release of the ring assembly from the insertion instrument.

FIG. 37C is a cross-sectional view of the distal portion of an anastomosis system depicted in FIG. 37A, after withdrawal of the insertion instrument.

FIG. 38A is a cross-sectional view of a further alternative embodiment of a distal portion of an anastomosis system, with a shaft flexing portion.

FIG. 38B is a cross-sectional view of the distal portion of an anastomosis system depicted in FIG. 38A, with the shaft flexing portion during flexing.

FIG. 38C is a cross-sectional view of the distal portion of an anastomosis system depicted in FIG. 38A, with the shaft flexing portion during further flexing.

FIG. 39 is a perspective view of the distal portion of a further alternative embodiment of an anastomosis system with the second ring assembly in the undeployed position.

FIG. 40 is a perspective view of an anastomosis system depicted in FIG. 39, with the second ring assembly in the partially deployed position.

FIG. 41 is a perspective view of an anastomosis system depicted in FIG. 39, with the second ring assembly in the fully deployed position.

FIG. 42 is a perspective view of a ring assembly of the alternative anastomosis system depicted in FIG. 39, in the fully deployed position.

FIG. 43 is a side view of a further alternative embodiment of an anastomosis system with the second central ring mounted proximally with respect to the second collar.

FIG. 44 is a perspective view of a further alternative embodiment of an anastomosis system with the second central ring mounted proximally with respect to the second collar.

FIG. 45 is a perspective view of a further exemplary embodiment of a first ring assembly structure for use with an anastomosis device.

FIG. 46 is a perspective view of a first exemplary embodiment of a second ring assembly structure for use with an anastomosis device.

FIG. 47 is a side view of the embodiments of the first ring assembly structure and the second ring assembly structure of FIGS. 45 and 46 deployed in tissue and connected together.

### DETAILED DESCRIPTION

The present disclosure generally relates to anastomosis systems and methods. In the depicted embodiments, the systems and methods relate to urethral anastomosis systems and methods. Persons of ordinary skill in the art will appreciate that the teachings herein can be readily adapted to other types of anastomosis systems and methods. Accordingly, as used herein, the terms such as urethra and bladder are not intended to be limiting of the embodiments of the present invention. Instead, it will be understood that the embodiments of the present invention relate generally to the field of medical devices and, in particular to devices and methods for connecting two hollow body parts or vessels, such as the urethra and the bladder, or portions of any other body vessel. As used herein, the terms “proximal” and “distal” refer respectively to the directions closer to and further from the operator of the anastomosis device. For purposes of clarity, the distal portion of the device is inserted furthest into an anastomosis patient and the proximal portion of the device remains closest to the inserting physician. Likewise, the term “lower” is generally used to refer to a proximal portion of the device, i.e. one that is proximally located with respect to a corresponding portion of the device. The term “upper” is generally used to refer to a distal portion of the device, i.e. one that is distally located with respect to a corresponding portion of the device. For frame of reference in the figures, arrows marked “P” refer generally to the proximal direction and arrows marked “D” refer generally to the distal direction relative to the orientation of the items depicted in the figures.

The anastomosis systems of the present disclosure generally include a coupling assembly for connecting and sealing the two body parts and a surgical implement for emplacing the coupling assembly. In typical embodiments, the coupling assembly includes two ring assemblies, with each ring assembly having securement elements that attach to the respective body part and interconnecting elements that attach to the other ring. For example, in some of the depicted embodiments for urethral anastomosis, the coupling assembly includes two ring assemblies each made of a degradable/absorbable material and interconnected to form a leak-proof seal between the bladder and the urethra. When used for urinary anastomosis, the coupling assembly, which may also be referred to as a ring assembly 3 herein, eliminates urine leakage, removing the cause of the most common post-operative complication, bladder-neck contracture. Also, the anastomosis is performed entirely within the urethra and thus there is no risk of damaging the neurovascular bundles that lie directly outside the urethra.

In addition, the surgical instrument of the anastomosis system can be used laparoscopically/robotically as well. Currently, a laparoscopic/robotic prostatectomy requires a hand-sewn urethral anastomosis that can take up to three hours and does not result in an immediate water-tight seal. There has been an enormous increase in robotic-assisted radical prostatectomies during the last five years. This surgical instrument can be used with the present coupling assembly to form a seal between the bladder and the urethra in only approximately fifteen minutes (rather than three hours) and the resulting seal is leak-proof. This system and method also presents the potential to perform the procedure without a urethral catheter, which is normally left in place within a patient for seven to ten days. Finally, the system and method will preferably only compromise about 4-8 mm of urethra, thereby maximizing “functional urethral length,” which is known to be one of the most important determinants of post-operative continence.

In the figures, in which like numerals indicate like elements throughout, there are shown exemplary embodiments of an anastomosis system. The first embodiment of the anastomosis system is generally referred to by the numeral 1.

### Ring Assembly

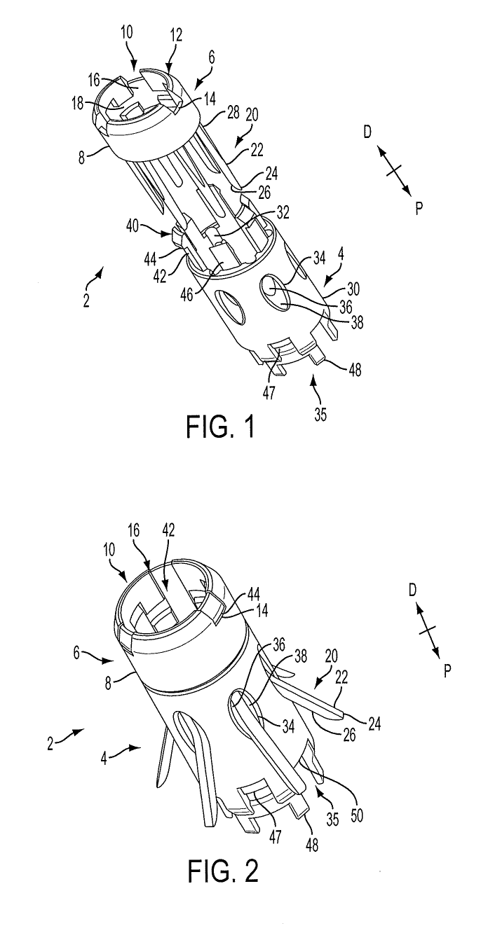

Turning now to the drawings, FIGS. 1 and 2 show a first ring assembly 2, which may be depicted as an upper or bladder ring assembly in certain applications of the device. In FIG. 1, the first ring assembly 2 is shown in the stored/retracted/delivery position. In FIG. 2, the first ring assembly 2 is shown in the deployed/extended position.

As shown in FIG. 1, the first ring assembly 2 comprises a first collar 4 and a first central ring 6. The first central ring 6 generally defines a ring shape having a first ring assembly wall 8 and lumen 10 that permits the passage of fluid therethrough. A distally facing surface 12 of the first ring assembly wall 8 defines locking tab receivers 14, which comprise indentations in the first ring assembly wall 8. The first ring assembly wall 8 facing the lumen 10 contains an axially extending device release groove 16 that communicates with a circumferentially extending deployment slot 18, along the interior of the first ring assembly wall 8. Additionally, the first central ring 6 has at least one first ring securement element 20, such as a tooth, extending axially in a proximal direction “P” from the first ring assembly wall 8 of the first central ring 6 opposite the distally facing surface 12. As shown, each first ring securement element 20 has an elongated body 22, a tissue piercing portion 24, and an inner surface 26. In FIG. 1, the elongated body 22 is generally straight, but may be curved so that the tissue piercing portions 24 are directed closer towards the lumen 10 of the first central ring 6.

In the depicted embodiment, the first ring securement elements 20 and the first central ring 6 are of a unitary construction. However, other constructions are possible. For example, the first ring securement elements 20 and the first central ring 6 may be separately constructed and the first ring securement elements 20 may each be pivotably mounted on the first central ring 6 so that the first central ring 6 forms a common axle for movement of the first ring securement elements 20 with respect to the first central ring 6.

As shown in FIG. 1, the first ring securement elements 20 are preferably formed from a resiliently flexible material that permits bending or flexing up to 30°, 90°, or 120° or any angle therebetween in a radial direction relative to the position shown in FIG. 1. The first ring securement elements 20 bend or flex from a stored/retracted/delivery position in which they extend axially from the first central ring 6 (as shown in FIG. 1) to a deployed/extended position in which they extend outward from the first collar 4 (as shown in FIG. 2) in order to engage and secure the first ring assembly 2 to tissue, such as the wall of the bladder neck or other hollow body part. Additionally, the first central ring 6 may be formed to include at least one living hinge (not shown) at a junction point 28 between at least one first ring securement element 20 and the first central ring 6. Alternatively, the deployment of the first ring securement elements 20 may rely on the flexibility and properties of the material forming the first ring securement elements 20 rather than a living hinge.

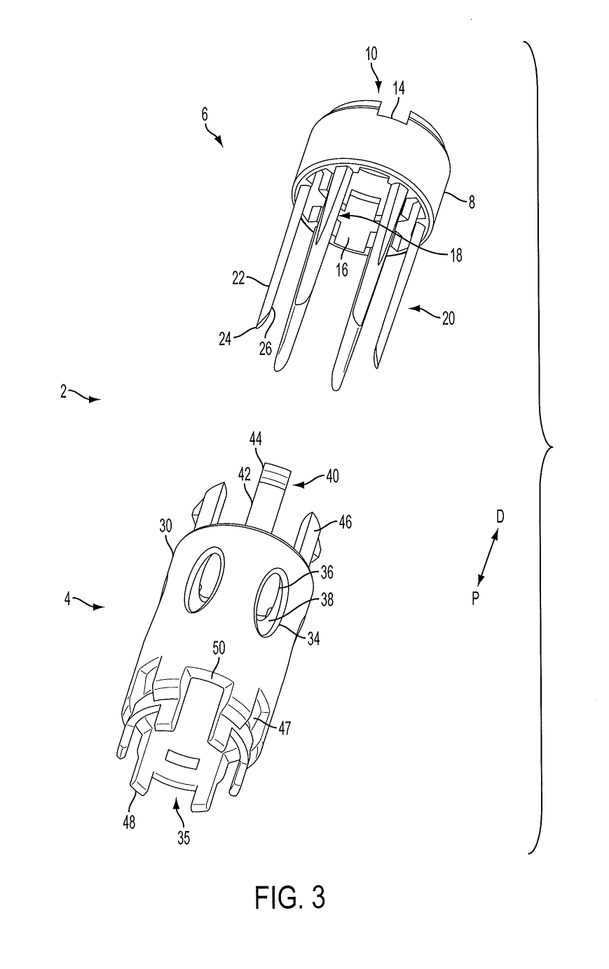

Referring to FIGS. 1 and 3, the first collar 4 is defined by a circumferential sidewall 30 comprising at least one axial groove 32 on its inner surface and at least one guide structure 34 in the sidewall 30. The first collar 4, defines a lumen 35 extending therethrough, which permits the passage of fluid through the first collar 4 and co-axially aligns with lumen 10 of the first central ring 6, when the first central ring 6 is mounted on the first collar 4. The axial grooves 32 extend axially along the interior surface of the circumferential sidewall 30 and are sized and shaped to guideingly receive a first ring securement element 20. The number and positioning of the axial grooves 32 correspond to the number and positioning of the first ring securement elements 20 such that each axial groove 32 may receive one first ring securement element 20.

The guide structures 34 are positioned in alignment with and proximally to the axial grooves 32. As shown in FIG. 1, the guide structures 34 define apertures 36 extending through the circumferential sidewall 30 of the first collar 4 that may extend at a proximally orientated angle with respect to the circumferential sidewall 30 of the first collar 4. The openings 36 of the guide structures 34 are sized and positioned to permit passage of the first ring securement elements 20 therethrough.

Still referring to FIGS. 1 and 3, each guide structure 34 defines an angled deployer surface 38 positioned to outwardly guide the first ring securement elements 20 as they pass through each aperture 36. When the first central ring 6 is mounted on the first collar 4, the first ring securement elements 20 extend through the internal lumen 6 of the first collar 4, into the axial grooves 32 and guide structures 34 such that a portion of the inner surfaces 26 of the first ring securement elements 20 engages the angled deployer surfaces 38. As shown in FIGS. 1 and 3, the number and positioning of the guide structures 34 correspond to the number and positioning of the first ring securement elements 20 such that each guide structure 34 may receive one first ring securement element 20.

Referring now to FIGS. 1 and 2, the first collar 4 further includes at least one ring mounting member 40 extending distally and axially from the first collar 4. Ring mounting members 40 include a ring wall receiving member 42 and a ring locking tab 44. The ring wall receiving member 42 is sized and configured to pass though the lumen 10 of the first central ring 6 and permit the first ring assembly wall 8 to be positioned between the circumferential sidewall 30 of the first collar 4 and the ring locking tab 44. As best seen in FIG. 2, when the first ring assembly wall 8 of the first central ring 6 is positioned between the circumferential sidewall 30 of the first collar 4 and a ring locking tab 44, (i) the ring locking tab 44 engages the locking tab receiver 14 of the first central ring 6 and (ii) the ring wall receiving member 42 is received in a extending device release groove 16. Engagement of the locking tab receivers 14 by the ring locking tabs 44 may restrict axial movement of the first central ring 6 with respect to the first collar 4, thereby securing the first central ring 6 and the first collar 4 together. As seen in FIG. 2, when the first central ring 6 and the first collar 4 are joined together, the first ring securement elements 20 fully project radially outward through the sidewall 30 of the first collar 4.

As best seen in FIG. 1, the first collar 4 also includes at least one ring guide 46 extending distally and axially from the circumferential sidewall 30 of the first collar 4. The ring guide 46 is a generally rectangular extension that may be received in the lumen 10 of the first central ring 6 to guide the mounting of the first central ring 6 onto the first collar 4. The ring guide 46 may be received within a groove or channel (not shown) in the first central ring 6 to guide mounting of the first central ring 6 onto the first collar 4. When the ring guide 46 is received in the groove or channel (not shown), the first ring securement elements 20 are aligned with guide structures 36 of the first collar 4 and rotational movement of the first central ring 6 with respect to the first collar 4 is restricted.

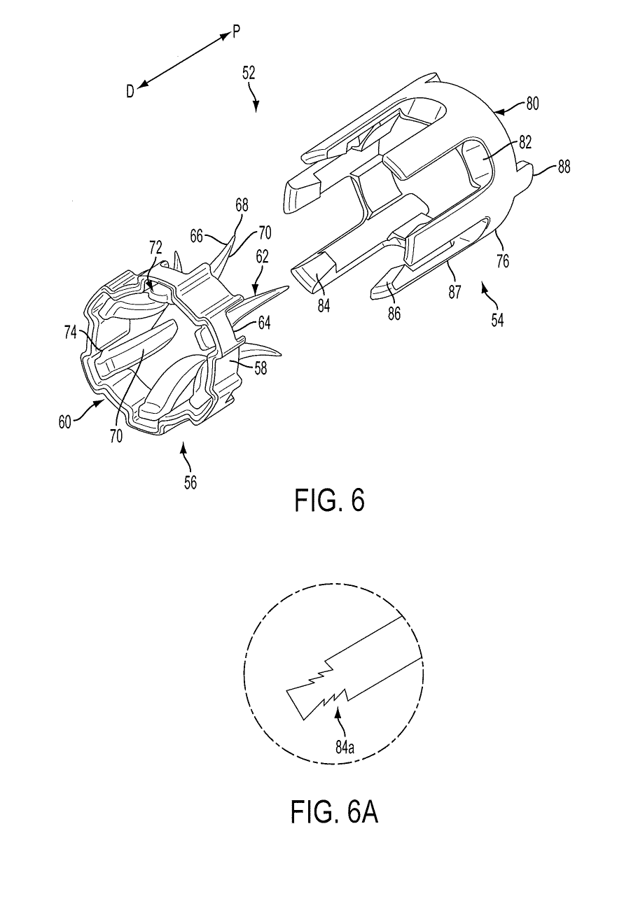

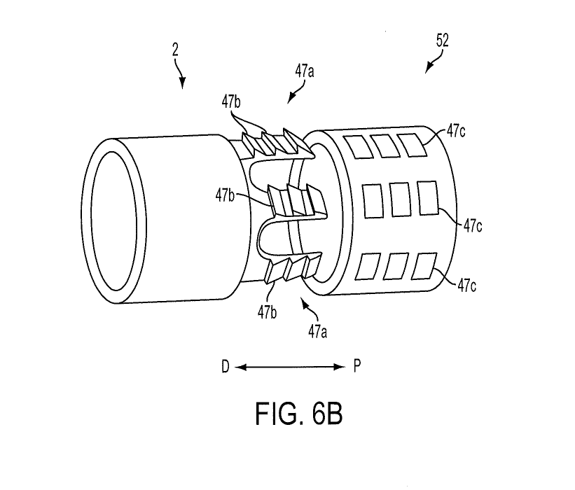

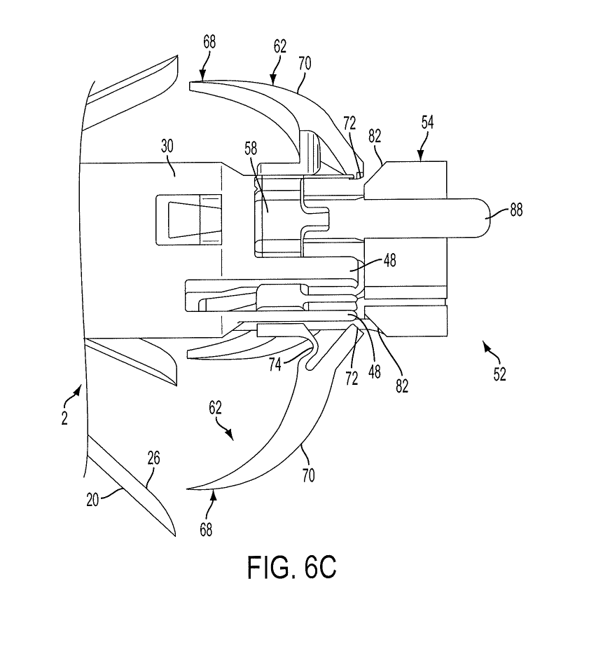

Turning now to the alternative view of the first collar 4 shown in FIG. 3, the first collar 4 is shown further including at least one first ring interconnecting element 47 proximally positioned on the first collar 4 for coupling the first collar 4 to the second collar 56 (shown in FIG. 6). The first ring interconnecting elements 47 can be provided as snap-fit connectors, screw-together connectors, adhesives or other conventional connector assemblies, whether detachable for decoupling or intended for one-time connection only. In typical embodiments, the first ring interconnecting elements 47 are provided by releasably interlocking catch surfaces that engage corresponding resiliently deflectable arms (such as second ring interconnecting elements 84 as depicted in FIG. 6), detents, push-pin assemblies, or other types of connectors for coupling two structures together.

In some examples, the first and second ring interconnecting elements 47, 84 may be configured to allow the ring assemblies 2, 52 to be selectively spaced apart from one another during coupling, for example, to accommodate variable length of the anastomosis or elasticity of the hollow body parts. For example, either or both of the first and second ring interconnecting elements 47, 84 may be provided with a plurality of notches, protuberances, or other coupling structures or means for coupling parts together (not shown in FIG. 2) that engage the opposing ring assembly to couple the first and second ring assemblies 2, 52 together. An example can be seen in FIG. 6A, where a second ring interconnecting element 84 includes multiple notches 84a for graduated attachment with the first ring assembly 2, via the first ring interconnecting element 47. Those skilled in the art will recognize that similar structures may also be provided on the first ring interconnecting assembly 47.

Another embodiment of ratcheting features that can be included on the first ring assembly 2 and second ring assembly 52 that are capable of providing a variable coupling distance between the first and second ring assemblies 2, 52 can be seen in FIG. 6B. As shown in FIG. 6B, the first ring assembly may include a plurality of interconnecting elements 47a that include a plurality of structures 47b that matingly engage corresponding interconnecting elements 47c included on the second ring assembly. Thus, in the embodiment shown in FIG. 6B, it is possible to couple the first and second ring assemblies 2, 52 together at three different distances. Thus, in the shown embodiment, the ring assemblies can be moved into contact with each other until the proximal-most structures 47b on the first ring assembly interconnecting elements 47a matingly engage the distal-most interconnecting elements 47c on the second ring assembly 52. Thus, in this position, the first and second ring assemblies 2, 52 are coupled together their farthest distance. If the surgeon desires to have a shorter coupling distance between the first and second ring assemblies 2, 52, the first and second ring assemblies 2, 52 may be moved closer together until the next-most structures 47b on the first ring assembly interconnecting elements 47a matingly engage the next-most interconnecting elements 47c on the second ring assembly 52. This process can continue until the desired coupling distance is achieved. In the depicted embodiment, the ratcheting features may be raised structures, detents, openings or any other structures that matingly engage each other to couple the first and second ring assemblies 2, 52 together. Although the depicted embodiment shows raised structures 47b on the first ring assembly interconnecting elements 47a and openings 47c in the second ring assembly 52 to receive the raised structures 47b, it is to be understood that the inclusion of these structures on the first and second ring assemblies 2, 52 may be reversed, i.e., the raised structures can be included on second ring assembly interconnecting elements.

The surgeon can manipulate the first and second ring assemblies 2, 52 so that a first notch or protuberance (not shown) or other similar structure on either or both the first and second ring interconnecting elements 47, 84 engages corresponding structures on the opposing ring assembly to couple the first ring assembly 2 at a first distance from the second ring assembly 52. If the first distance between the ring assemblies 2, 52 is determined to be too close or too far, the surgeon can manipulate the first and second ring assemblies 2, 52 so that a different notch or protuberance (not shown) or other similar structure on either or both the first and second ring interconnecting elements 47, 84 engages a corresponding structure on the opposing ring assembly to couple the first ring assembly 2 at a second distance from the second ring assembly 52. Those skilled in the art will recognize that adjusting the distance between the first and second ring assemblies 2, 52 can be performed numerous times until the desired distance between the two ring assemblies and hence, the desired magnitude of contact between the body tissue to be joined or connected, is obtained.

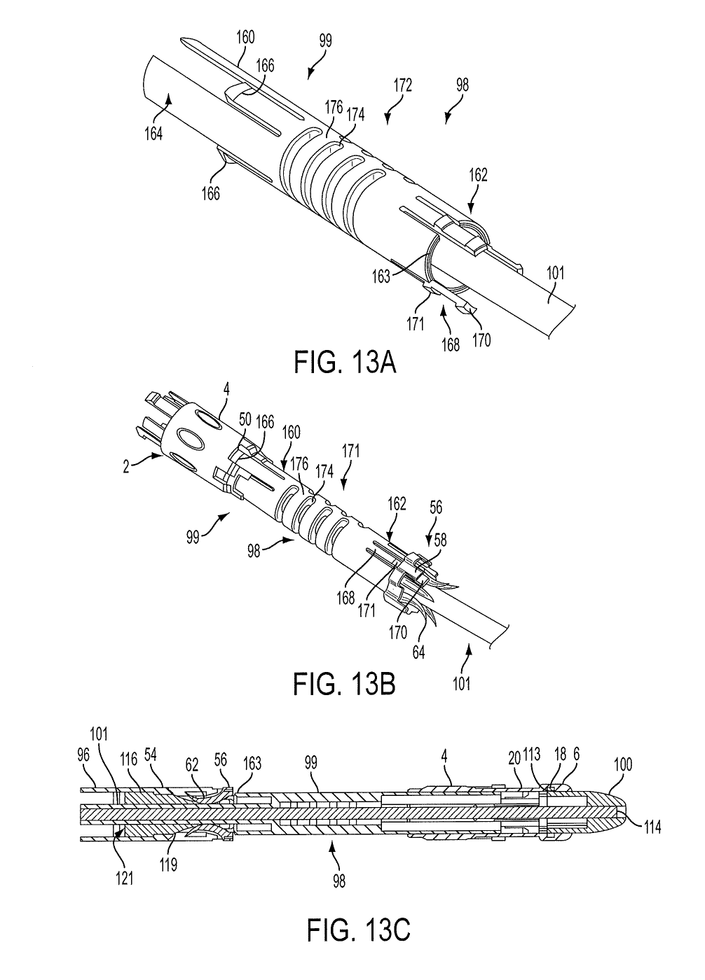

The first collar 4 further includes at least one proximally and axially extending second ring securement element locking member 48 for locking the second ring securement elements 62 of the second ring assembly 52 (shown in FIGS. 6 and 6C) in the deployed position when the first ring assembly 2 and second ring assembly 52 are coupled together (discussed in further detail with respect to FIGS. 6 and 6C). As shown, the second ring securement element locking member 48 extends proximally from the circumferential sidewall 30 of the first collar 4 adjacent to the support surfaces 50. The second ring securement element locking members 48 are preferably tapered from a thinner portion at its tip towards its thickest portion adjacent to the upper collar sidewall 30 to further assist in guiding the alignment and coupling of the ring assemblies 2, 52 together. There may also be additional taper provided to the side of each second ring securement element locking members 48 to help align the first and second ring assemblies about their longitudinal axis, if necessary. The second ring securement element locking member 48 serves to restrict rotation of first and second ring assemblies 2, 52 with respect to each other when the ring assemblies 2, 52 are coupled together, but preferably does not restrict axial movement. Instead, the lower ring interconnecting element 47 may help to limit unintended axial movement of the first ring assembly 2 with respect to the second ring assembly 52. The support surfaces 50 are proximally facing surfaces extending generally perpendicular to circumferential sidewall 30 of the first collar 4. As discussed further with respect to FIG. 13B, the support surfaces 50 facilitate the mounting of the first collar 4 for deployment.

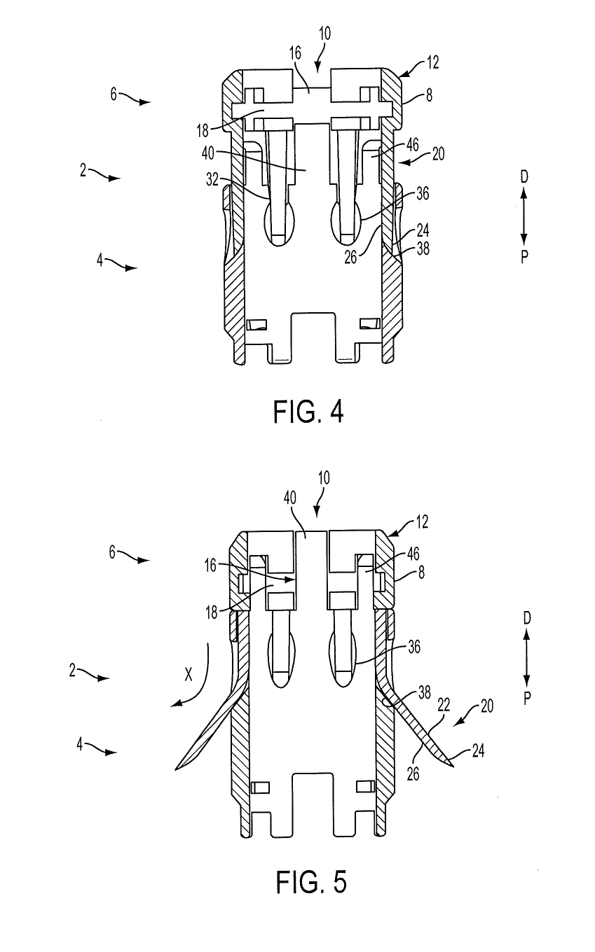

Referring now to FIGS. 4 and 5, the first central ring 6 is mounted on the first collar 4 with the first ring assembly 2 in the retracted/stored position (FIG. 4) and the extended/deployed position (FIG. 5). As shown in FIG. 4, when the first ring assembly 2 is in the retracted or undeployed position, the first central ring 6 is spaced distally with respect to the first collar 4 such that the first ring securement elements 20 are received in axial grooves 32 and openings 36 and the tissue piercing portions 24 are directed towards the angled deployer surface 38. In this position, the first ring securement elements 20 are received by the first collar 4 such that the first ring securement elements 20 extend axially from the first central ring 6 in the proximal direction without substantially bending or flexing. Thus, in this position, the tissue piercing portions 24 do not engage body tissue.

FIG. 5 shows that movement of the first central ring 6 towards the first collar 4 during deployment urges the tissue piercing portions 24 and inner surfaces 26 of the first ring securement elements 20 against the angled deployer surfaces 38 of the first collar 4. Further translation or movement of the first central ring 6 towards the first collar 4 or vice versa, translation or movement of the first collar 4 towards first central ring 6, urges the first ring securement element body 22 to bend or flex where the first ring securement element 20 contacts the angled deployer surface 38 such that the first ring securement element 20 extends proximally and radially outward from the first collar 4 (as illustrated by arrow “x” in FIG. 5). Additionally, during translation or movement of the first central ring 6 towards the first collar 4 or vice versa, translation or movement of the first collar 4 towards first central ring 6, the ring mounting member 40 and the ring guide 46 may extend into the lumen 10 of the first central ring 6 and engage the inner surface of the first ring assembly wall 8. Where translation or movement of the first central ring 6 towards the first collar 4 or, vice versa, translation or movement of the first collar 4 towards first central ring 6, brings the first ring assembly wall 8 into contact with the circumferential sidewall 30 of the first collar 4, the ring locking tab 44 may engage the locking tab receiver 14 (as best seen in FIGS. 1 and 2). Engagement of the ring locking tab 44 with the locking tab receiver 14 may assist in restricting translational and/or rotational movement of the first central ring 6 with respect to the first collar 4, thus retaining the first ring securement elements 20 in the deployed position and also joining the upper collar 4 and upper central ring 6 together.

Turning now to FIGS. 6 and 6C, an exemplary second (e.g., lower or urethra) ring assembly 52 having a second collar 54 and a second central ring 56 is shown. The second central ring 56 has a second ring assembly wall 58 generally defining a lumen 60 extending therethrough, which permits the passage of fluid through the second central ring 56. At least one second ring securement element 62 is mounted on a second ring securement element mounting member 64 that defines a radially extending portion of the second ring assembly wall 58. Each of the second ring securement elements 62 extend axially along the lumen 60 of the second central ring 56. As shown, each second ring securement element 62 has a curved body 66, a tissue piercing portion 68, and an inner surface 70. In alternate embodiments, the second ring securement elements 62 may have a straight body. The second ring securement elements 62 also have a second ring securement element cam surface 72 opposite the piercing tip 68 and a pivot point 74.

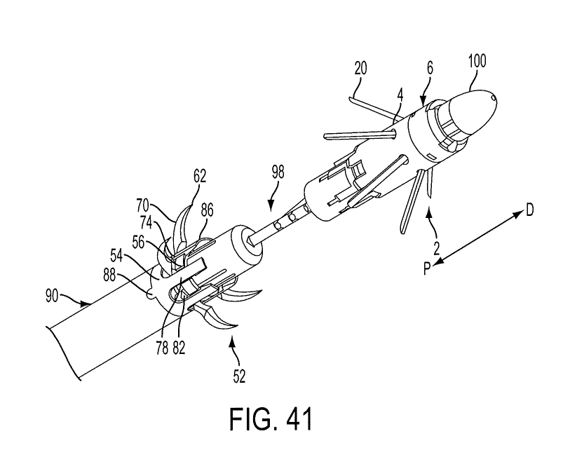

As shown, the second ring securement elements 62 and the second central ring 56 are made of a unitary construction. The second ring securement elements 62 are adapted to bend, flex or rotate about a pivot point 74 from a stored/retracted/delivery position, in which they extend axially from the second central ring 56 through the lumen 60 (as shown in FIG. 6) to a deployed/extended position, in which they extend outward from the second central ring 56 (as best shown in FIGS. 6C, 29D, 30D, 31D, 41 and 42), such that the second ring securement elements 62 engage and secure the second ring assembly 52 to body tissue, such as the wall of the urethra neck or other hollow body part. In some examples, the pivot point 74 may comprise a living hinge; however, other structures are possible. For example, the second ring securement elements 62 and the second central ring 56 may be separately constructed and the second ring securement elements 62 may each be pivotably mounted on the second central ring 56 so that the second central ring 56 forms a common axle.

Still referring to FIG. 6, the second collar 54 is shown having a proximal ring base 76 and at least one longitudinally extending member 78 defining a lumen 80. The longitudinally extending members 78 extend axially and distally from the proximal ring base 76 and are spaced apart to slideably receive a second ring securement element mounting member 64 therebetween. Between each longitudinally extending member 78 is a distally facing surface of the proximal ring base 76 which defines an angled second ring securement element engagement surface 82. The second ring securement element engagement surface 82 is angled to engage the inner surface 70 of the second ring securement element 62 and deflect the second ring securement elements 62 outwards when the second central ring 56 is translated or moved towards the second collar 54 or, vice versa, the second collar 54 is translated or moved towards the second central ring 56.

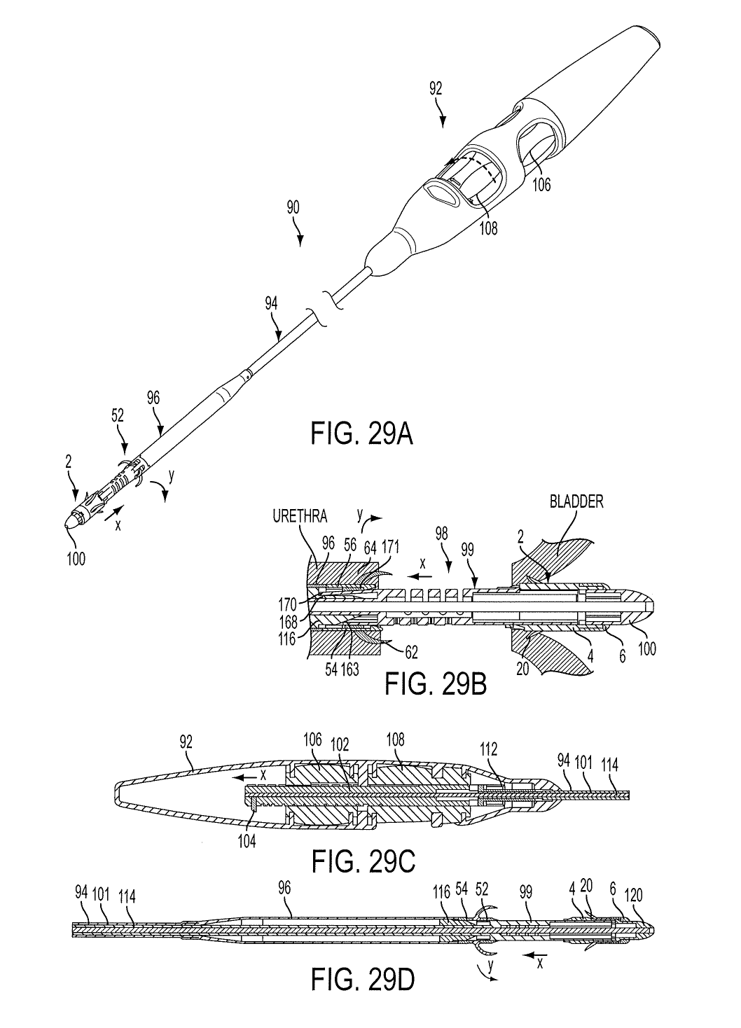

As shown in FIG. 6, a second ring interconnecting element 84 is positioned distally on at least one of the longitudinally extending members 78 opposite the proximal ring base 76. The second ring interconnecting element 84 defines a protrusion extending into the lumen 80 and is configured to engage the first ring interconnecting element 47 and couple the second ring assembly 52 and first ring assembly 2 together when the second ring assembly 52 and first ring assembly 2 are urged towards mutual contact, as best seen, for example, in FIGS. 29D, 30D, 31D, and 42. The second ring interconnecting elements 84 can be snap-fit connectors, screw-together connectors, adhesives, or other conventional connector assemblies, whether detachable for decoupling or intended for one-time connection only. Additionally, a second central ring lock 86 is positioned distally on a shorter longitudinally extending member 87. The second central ring lock 86 includes a protrusion extending into the lumen 80 and is configured to engage the second central ring 56 when the second central ring 56 is received in the second collar 54, thereby allowing the second central ring 56 to be retained proximally of the first ring assembly 2, when the ring assemblies 2, 52 are deployed and attached to each other. A plurality of second central ring locks 86 and shorter longitudinally extending members 87 may be included. Alternatively, the second central ring 56 may be held in place within the second collar 54 by a friction fit. In any event, once the first ring assembly 2 and second ring assembly 52 are coupled together, this coupling will lock the second central ring 56 in place within the second collar 54.

Similar to the disclosure above with respect to FIG. 6A, the second central ring lock 86 may be provided with one or more notches (not shown) or similar structures that allow the surgeon to selectively couple the first ring assembly 2 with more or less proximity to the second ring assembly 52. Thus, the one or more notches or similar structures may serve as a ratcheting mechanism (not shown) that allows the surgeon to adjust the proximity of the first and second ring assemblies 2, 52 to accommodate the length or elasticity of the hollow body parts. Additionally or alternatively, the ratcheting mechanism (not shown) may be provided by one or more notches or similar structures provided on the first ring interconnecting element 47. Those skilled in the art will recognize that adjusting the distance between the first and second ring assemblies 2, 52 can be performed numerous times until the desired distance between the two ring assemblies and hence, the desired magnitude of contact between the body tissue to be joined or connected, is obtained.

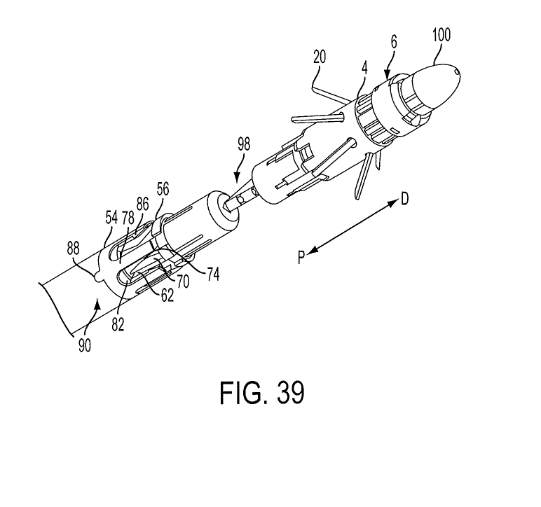

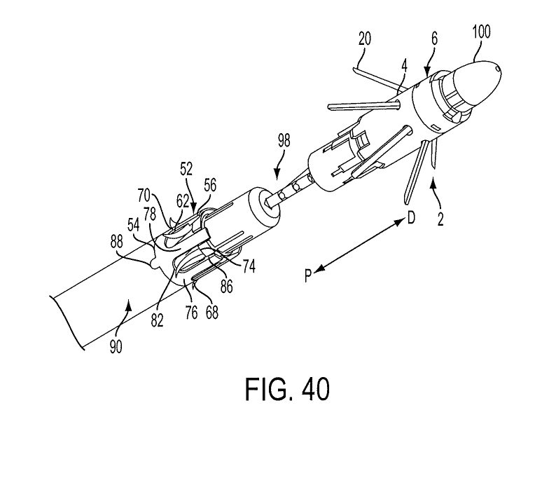

Referring to FIG. 39, the second collar 54 is configured to receive the second central ring 56 when the second central ring 56 is translated or moved towards the second collar 54, or vice versa, the second collar 54 is translated or moved towards the second central ring 56, such that the second ring securement element mounting members 64 and second ring securement elements 62 slide between adjacent extending members 78. As shown in FIG. 40, when the second central ring 56 slides proximally towards the proximal ring base 76 and past the second central ring lock 86, the second central ring lock 86 restricts translation of the second central ring 56 away from the second collar 54. Further advancement of the second central ring 56 into sliding engagement with the second collar 54 results in engagement of the inner surfaces 70 of the second ring securement elements 62 with the angled second ring securement element engagement surfaces 82 of the second collar 54. Engagement of the second ring securement elements 62 with the angled second ring securement element engagement surface 82 displaces the second ring securement elements 62 outwardly from the longitudinal axis of the second central ring 56, thereby urging the second ring securement elements 62 to pivot around a pivot point 74 and extend outward towards the partially deployed position.

As shown in FIG. 40, in the partially deployed position (as best seen in FIG. 40), the tissue piercing portions 68 of the second ring securement elements 62 extend outward in a generally proximal direction to pierce and engage the second hollow body part, such as the urethra. However, in the partially deployed position, the second ring securement elements 62 may not securely engage the second hollow body part so as to substantially restrict distal translation of the second central ring 56 with respect to the second hollow body part.

Furthermore, in the partially deployed position, a portion of the second ring securement element cam surface 72 extends into the lumens 60, 80 of the second central ring 56 and second collar 54. Additional force in the proximal direction applied to the second ring securement element cam surface 72 of the second ring securement elements 62 drives the second ring securement elements 62 towards full deployment (also shown in FIGS. 29A, 29B, 41, and 42). The second ring securement elements 62 pivot around a pivot point 74 from the undeployed position, such that the second ring securement element cam surfaces 72 are substantially axially aligned with the second ring securement element mounting member 64. In the fully deployed position (as shown in FIGS. 29D, 30D, 31D, 41, and 42), the second ring securement elements 62 may extend outward in a generally lateral direction and securely engage body tissue or a vessel such as the urethra , so as to substantially restrict translation or movement of the second ring assembly 52 with respect to the second hollow body part (e.g., urethra). Additionally, the tissue piercing portions 68 of the second ring securement elements 62 may be directed towards the second collar 54, as opposed to being pointed radially outward, into the surrounding tissue, thus minimizing damage to the surrounding tissue when the ring assembly 3 is in place.

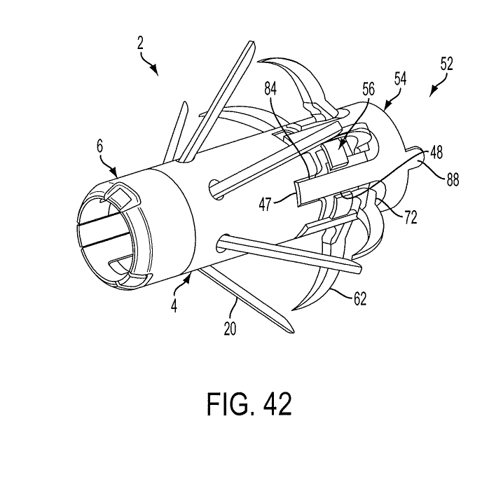

Referring now to FIGS. 6C and 42, when the second ring assembly 52 and first ring assembly 2 are both fully deployed and brought into interlocking engagement, the second ring securement element cam surfaces 72 cooperate with the second ring securement element locking members 48 of the first collar 4 to lock the second ring securement elements 62 in the fully deployed position. When the second ring assembly 52 and first ring assembly 2 are urged towards interlocking engagement, the first ring assembly 2 and second ring assembly 52 are in axial alignment such that the second ring securement element locking members 48 of the first collar 4 extend into the lumen 60 of the second central ring 56. During coupling of the first ring assembly 2 and second ring assembly 52, the second ring securement element locking member 48 slide against the lumen-facing surface of the second ring securement element mounting members 64 and the second ring securement element cam surfaces 72 (which are axially aligned with the second ring securement element mounting members 64 in full deployment). The positioning of the second ring securement element locking member 48 within the lumen 60 and in contact with the second ring securement element cam surfaces 72 restricts movement of the second ring securement element cam surfaces 72 into the lumen 60, thereby locking the second ring securement elements 62 in the fully deployed position as shown in FIG. 6C.

Referring now to FIG. 6, at least one instrument engaging element 88 is provided on the second collar 54. The instrument engaging element 88 is a protrusion extending proximally from the proximal ring base 76 of the second collar 54 that engages an instrument 90 (shown in FIGS. 39-41) by friction fit, press fit, compression fit, or other attaching means. The instrument engaging element 88 restricts rotation of the second ring assembly 52 with respect to the insertion instrument 90 and proximal translation of the second collar 54 with respect to the insertion instrument 90. However, the instrument engaging element 88 is adapted to facilitate release of the second collar 54 from the insertion instrument 90 when the second ring assembly 52 is secured to the second hollow body part (e.g., urethra) and the insertion instrument 90 is translated proximally away from the second ring assembly 52.

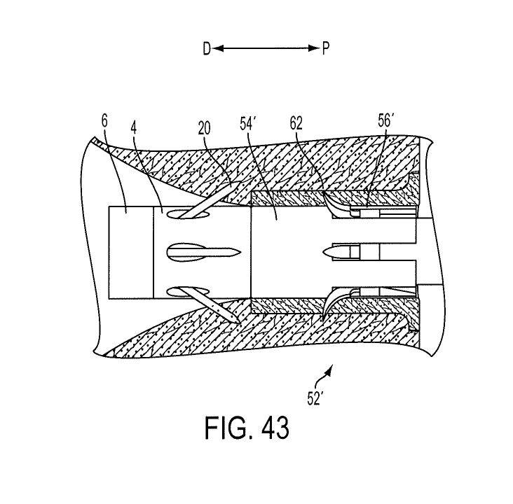

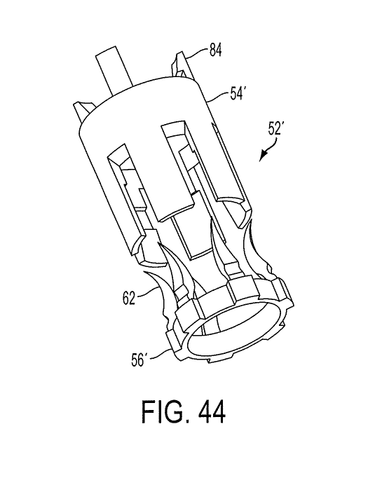

Referring now to FIGS. 43 and 44, a slightly modified alternative embodiment of the deployment of the second ring assembly 52′ is shown. As shown in FIG. 43, the second central ring 56′ may be mounted adjacent to the second collar 54′ on an opposite side of the second collar 54′ than the embodiment shown in FIG. 6. In the embodiment shown in FIG. 43, the second ring assembly 52′ may be deployed by translation or movement of the second central ring 56′ distally towards the second collar 54′. Additionally, as shown best in FIG. 44, an embodiment of a second ring assembly 52′ having the second central ring 56′ may be mounted proximally with respect to the second collar 54′ and may also be provided with second ring interconnecting elements 84 positioned distally on the second collar 54′.

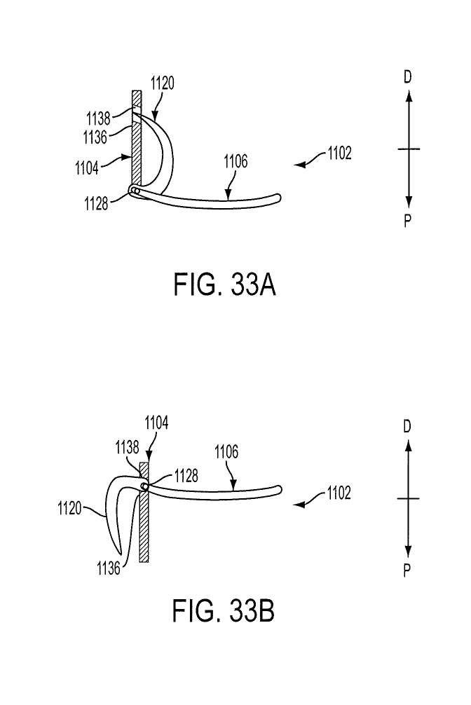

One skilled in the art will appreciate that alternate embodiments of a ring assembly 3 are possible, such as the alternative exemplary embodiment of a first ring assembly 1102 depicted in FIGS. 33A and 33B. Like the embodiment of a first ring assembly 2 shown in FIG. 1, the first ring assembly 1102 includes a first collar 1104 and a first central ring 1106. As shown, the first central ring 1106 may be of a unitary construction with the first ring securement elements 1120, and the first ring securement elements 1120 may be mounted on the first central ring 1106. Although a single first ring securement element 1120 is shown here for illustrative purposes, multiple first ring securement elements 1120 may be mounted to the same first central ring 1106. Unlike the embodiment of the first central ring 6 shown in FIG. 1, the first central ring 1106 shown in FIGS. 33A and 33B may be configured to rotate or evert during deployment of the first ring securement elements 1120.

As shown in FIGS. 33A and 33B, the distal translation or movement of the first central ring 1106, with respect to the first collar 1104, or vice versa, the proximal translation or movement of the first collar 1104 with respect to the first central ring 1106, urges the first ring securement elements 1120 into contact with the guide structures 1138 of the first collar 1104. The force of the first ring securement elements 1120 against the guide structures 1138 of the first collar 1104 urges the first ring securement elements 1120 to pivot at and translate through the rotation of the first central ring 1106 about itself. The first central ring 1106 is sufficiently flexible to allow eversion wherein an inner facing surface is positioned to face outwards and an outward facing surface is positioned to face inwards. Accordingly, the pivoting motion of the first ring securement elements 1120 causes the first central ring 1106 to also rotate and evert. As shown in FIGS. 33A and 33B, the dots on the first central ring 1106 rotate from an upward direction shown in FIG. 33A to a downward direction shown in FIG. 33B as the first central ring 1106 rotates and everts. Optionally, the first central ring 1106 may comprise living hinges 1128 used to mount the first ring securement elements 1120 and reduce the overall stress on the first ring securement elements 1120 by allowing the first central ring 1106 to rotate. As a result, the stress concentration at the living hinge 1128 is reduced, thus reducing the chance of failure at the living hinge during deployment. Additionally, there may be cam structures or ratcheting teeth (not shown) on the back of the securement elements. In preferred examples, a stop mechanism is a tooth (not shown) on the central ring 1106 that rotates 180 degrees within the collar 1104 and then abuts an internal structure on the inner wall of the collar 1104 to resist rotation of the first central ring 1106 back to the undeployed position. Additionally, one skilled in the art will appreciate that a structure similar to FIGS. 33A and 33B may be adapted for use a second ring assembly (not shown) for engagement and securement to the urethra or other hollow body part.

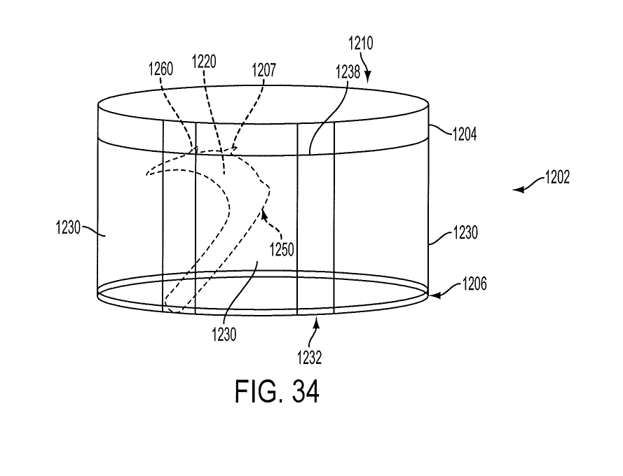

Additionally, a further alternative embodiment of a first ring assembly 1202 is depicted in FIG. 34. As shown, the first ring assembly 1202 is defined by a circumferential sidewall, which is made up of multiple panels 1230 that attach to a first ring structure 1204 and a second ring structure 1206, thereby defining the circumferential wall of the first ring assembly 1202. Preferably, the panels 1230 are formed from a flexible and elastic fabric, polymer sheeting, or other material so long as the material is flexible and elastic.

As also shown in FIG. 34, the panels 1230 are arranged about the circumference of the first ring assembly 1202 such that axially extending slots 1232 separate each panel. Each of the axially extending slots 1232 is sized and spaced to receive a first ring securement element 1220, which are pivotably mounted on the second ring structure 1206. The circumferential sidewall further defines guide surfaces 1238 positioned distally in the axially extending slots 1232 on the first ring structure 1204. In alternate embodiments, the circumferential sidewall may be made from a single flexible and elastic material attached to the first ring structure 1204 and second ring structure 1206. In such embodiments, the axially extending slots may be cut into the flexible and elastic material.

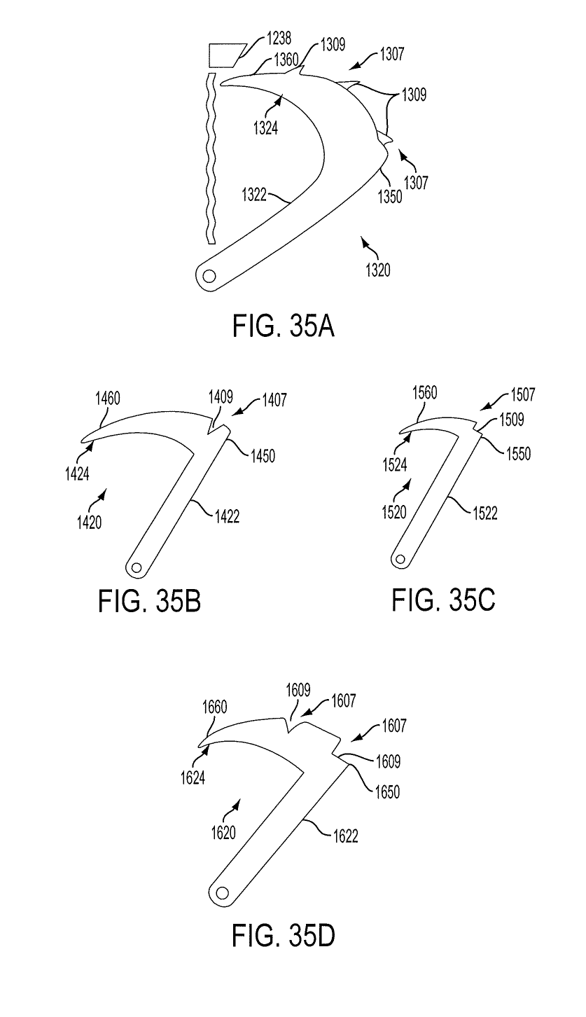

As shown in FIG. 34, the first ring securement elements 1220 may define at least one ratcheting element 1207 (or means for adjusting the positioning of the first ring securement elements 1220 with respect to the circumferential sidewall) positioned to engage the guide surface 1238 of the first ring structure 1204 during deployment of the first ring assembly 1202. As best seen in the exemplary embodiments of alternative first ring securement elements (1320, 1420, 1520, 1620) shown in FIGS. 35A-35D, the first ring securement elements 1320, 1420, 1520, 1620 may define a bent or sickle-shaped body 1322, 1422, 1522, 1622 with a curved tissue piercing portion 1224, 1324, 1424, 1524, 1624. As shown, the tissue piercing portion 1224, 1324, 1424, 1524, 1624 is provided with a ratcheting element 1207, 1307, 1407, 1507, 1607 in proximity to the piercing tip of the securement element. As shown in FIG. 35A, a ratcheting element 1307 may be defined by at least one tooth 1309 extending from the tissue piercing portion 1324 of the first ring securement element 1320. Alternatively, as shown in FIGS. 35B-35D, a ratcheting element 1407, 1507, 1607 may be defined by at least one notch 1409, 1509, 1609 in the tissue piercing portion 1424, 1524, 1624. In alternate embodiments, the first ring securement elements may include multiple teeth or notches.

Referring again to FIG. 34, when the panels 1230 are in the unflexed or unstressed state, the distance between the first and second ring structures 1204, 1206 and hence the height of the axially extending slots 1232, is less than the height of the first ring securement elements 1220 such that the first ring securement elements 1220 are prevented from extending through the slots 1232 and are, therefore, maintained within the diameter of the first ring assembly 1202. Thus, in order to deploy the first ring securement elements 1220 through the axially extending slots 1232 and into body tissue, portions of the insertion instrument are brought into contact with the interior surface 1250, 1350, 1450, 1550, 1650 of the securement elements 1220, 1320, 1420, 1520, 1620. Further pressure or force exerted by the insertion instrument on the interior surfaces 1250, 1350, 1450, 1550, 1650 of the securement elements 1220, 1320, 1420, 1520, 1620 in a direction away from the longitudinal axis of the first ring assembly 1202, forces the securement elements 1220, 1320, 1420, 1520, 1620 to move in a corresponding direction into the axially extending slots 1232 such that a top surface 1260, 1360, 1460, 1560, 1660 of the first securement elements 1220, 1320, 1420, 1520, 1620 acts on the first ring structure 1204. Because the panels 1230 are made from a flexible and elastic material, as the first securement elements 1220, 1320, 1420, 1520, 1620 are further forced into axially extending slots 1232 by the insertion instrument, the shape of the top surface 1260, 1360, 1460, 1560, 1660 of the first securement elements 1220, 1320, 1420, 1520, 1620 forces the first ring structure 1204 away from the second ring structure 1206 thereby increasing the distance between the first and second ring structures 1204, 1206 and hence the length or height of the axially extending slots 1232. The increased length or height of the axially extending slots 1232 permits the first securement elements 1220, 1320, 1420, 1520, 1620 to enter into and through the axially extending slots 1232. The insertion instrument may push the first securement elements 1220, 1320, 1420, 1520, 1620 outwardly causing them to extend through the axially extending slots and into body tissue until a tooth 1309 or a notch 1409, 1509, 1609 catches on the first ring structure 1204. Once a tooth 1309 or a notch 1409, 1509, 1609 catches on the first ring structure 1204, tension on the first ring structure as a result of the flexible and elastic material of the panels 1230 acts to lock the first securement elements 1220, 1320, 1420, 1520, 1620 in the deployed position.

Moreover, because the panels 1230 and hence the material that forms the sidewall are made from a flexible and elastic material, after the first securement elements 1220, 1320, 1420, 1520, 1620 are deployed and held in place by the interaction of the ratcheting elements 1207, 1307, 1407, 1507, 1607 with the first ring structure 1204, the distance between the first ring structure 1204 and second ring structure 1206 can be increased because of the ability of the flexible and elastic material to stretch. Once the distance between the first and second ring structures 1204, 1206 is increased a sufficient amount, the ratcheting elements 1207, 1307, 1407, 1507, 1607 will disengage from the first ring structure 1204 allowing the first securement elements 1220, 1320, 1420, 1520, 1620 to retract within the circumference of the first ring assembly 1202 thereby permitting the surgeon to reposition the first ring assembly 1202 within the body vessel. This process can be repeated multiple times until the first ring assembly 1202 is properly positioned.

As shown in FIGS. 35A and 35D, in alternate embodiments, the ratcheting element may include multiple teeth 1309 (FIG. 35A) or multiple notches 1609 (FIG. 35D) such that the first securement elements 1220, 1320, 1420, 1520, 1620 may be extended outwardly through the axially extending slots 1232 at differing degrees depending on how much body tissue penetration the surgeon desires.

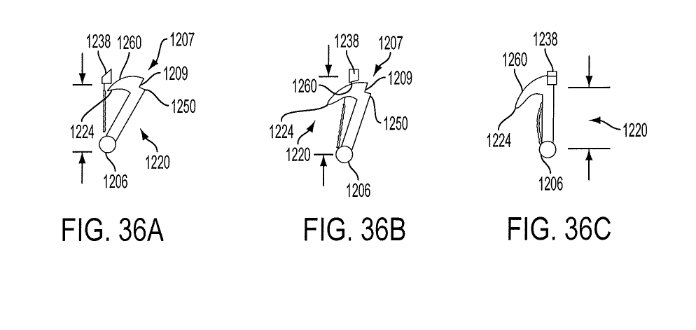

FIGS. 36A-36C depict an exemplary deployment procedure for first ring securement elements 1320, 1420, 1520, 1620 being provided with a notch 1409, 1509, 1609 or a tooth 1309, where the notch 1409, 1509, 1609 or tooth 1309 engages the guide surface 1238 of the first ring structure 1204 when the first ring securement element 1320, 1420, 1520, 1620 pivots radially with respect to the second ring structure 1206. Engagement of the notch 1209 with the guide surface 1238 causes the ratcheting element 1207 to restrict further pivoting movement of the first ring securement element 1220 with respect to the second ring structure 1206. The ratcheting element 1207 can be released to allow further pivoting movement of the first ring securement elements 1220 with respect to the first central ring 1206 by stretching of the panels 1230 in distal and/or proximal directions. Release of the ratcheting element 1207 may permit the first ring securement elements 1220 to retract towards the undeployed position or, in embodiments having a ratcheting element 1207 with plurality of teeth 1209, to pivot outwards until the guide structure 1238 engages a second tooth 1209.

One skilled in the art will appreciate that the alternative embodiments of the first ring assembly 1102, shown in FIGS. 33A and 33B, and the first ring assembly 1202, shown in FIG. 34, can also be utilized in a second ring assembly (not shown) or be used interchangeably with the design for ring deployment shown in FIGS. 1-6. One skilled in the art will further appreciate that any of the above disclosed ring assemblies can be used or modified for use in engaging and securing tissue, such as either of the bladder and the urethra, or any other hollow body part.

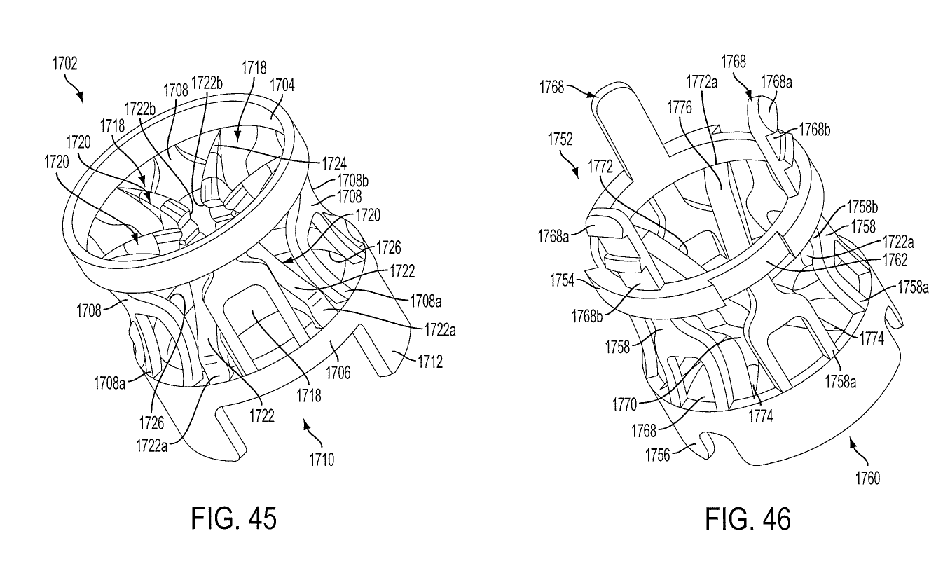

A further alternative embodiment of a first ring assembly 1702 is depicted in FIG. 45. The first ring assembly 1702 comprises a first upper ring 1704 and a first lower ring 1706 spaced proximally from and joined to the first upper ring 1704 by a plurality of strut assemblies 1708 extending therebetween. The first upper and lower rings 1704, 1706 generally define a circular structure having a lumen 1710 that permits the passage of fluid therethrough. The strut assemblies 1708 are spaced circumferentially around the first ring assembly 1702 and define spaces or apertures 1718 between each strut assembly 1708. The strut assemblies 1708 may be wishbone shaped having a pair of leg portions 1708a, adjoining the first lower ring 1706 and extending distally toward the first upper ring 1704, and a single central post 1708b extending generally proximally from the first upper ring 1704 and joining the leg portions 1708a to form the strut assembly 1708. The center of the strut assemblies 1708 may also be curved inward towards the lumen 1710 as shown in FIG. 45 or, alternatively, extend longitudinally between the first upper and lower rings 1704, 1706.

The first ring assembly 1702 has a plurality of securement elements 1720, each having an elongated body 1722, a tissue piercing portion 1724, and an inner surface 1726. As shown in FIG. 45, the securement elements 1720 are in the undeployed position, wherein the tissue piercing portions 1724 are disposed radially within the outer circumference of the first ring assembly, thereby facilitating insertion of the device to a desired location as contemplated herein. The tissue piercing portion 1724 preferably has an angled or hooked profile, adapted to facilitate retention of tissue as the securement element 1720 is deployed and the first ring assembly 1702 is moved in a generally proximal direction. A proximal portion 1722a of each elongated body 1722, opposite the tissue piercing portion 1724, is flexibly connected (e.g., through a living hinge) to the first lower ring 1706, thereby facilitating movement of the securement elements 1720 from an undeployed position (as depicted in FIG. 45), through the apertures 1718, to a deployed position. During deployment of the device, an internal cam or press mechanism may engage the inner surface 1726, thereby facilitating radially outward movement of the tissue piercing portions 1274 via pivoting of the elongated body 1722 about its connection point 1722a to the first lower ring 1706. The first ring assembly 1702 and securement elements 1720 contained therein may be deployed in accordance with the deployers and techniques disclosed herein, or via some other means that forces the securement elements 1720 radially outward of the first upper ring 1704. The distal portion of the elongated body 1722, which supports and engages the tissue piercing portion 1724, is configured with a deployment locking mechanism, such as a locking tab 1722b that is adapted to maintain the securement element 1720 in the deployed position (not shown) by engaging the first upper ring 1704 and resisting radially inward movement of the tissue piercing portion 1724, after deployment.

A further alternative embodiment of a second ring assembly 1752 is depicted in FIG. 46. The second ring assembly 1752 comprises a second upper ring 1754 and a second lower ring 1756, spaced proximally from and joined to the second upper ring 1754 by a plurality of strut assemblies 1758 extending therebetween. Like the first ring assembly 1702, the second upper and lower rings 1754, 1756 generally define a circular structure having a lumen 1760 that permits the passage of fluid therethrough. The strut assemblies 1758 are spaced circumferentially around the second ring assembly 1752 and define spaces or apertures 1768 between each strut assembly 1758. Also like the first ring assembly 1702, the strut assemblies 1758 may be wishbone shaped having a pair of leg portions 1758a, adjoining the first lower ring 1756 and extending distally toward the first upper ring 1754, and a single central post 1758b extending generally proximally from the first upper ring 1754 and joining the leg portions 1758a to form the strut assembly 1758. The center of the strut assemblies 1758 may also be curved inward towards the lumen 1760 or, alternatively, extend longitudinally between the first upper and lower rings 1754, 1756.

The second ring assembly 1752 has a plurality of securement elements 1770, each having an elongated body 1772, a tissue piercing portion 1774, and an inner surface 1776. As shown in FIG. 46, the securement elements 1770 are in the undeployed position, wherein the tissue piercing portions 1774 are disposed radially within the outer circumference of the second ring assembly 1752, thereby facilitating insertion of the device to a desired location as contemplated herein. A proximal portion 1772a of each elongated body 1772, opposite the tissue piercing portion 1774, is flexibly connected (e.g., through a living hinge) to the second upper ring 1756, thereby facilitating movement of the securement elements from an undeployed position (as depicted in FIG. 46), through the apertures 1768, to a deployed position. During deployment of the device, an internal cam or press mechanism may engage the inner surface 1776, thereby facilitating radially outward movement of the tissue piercing portions 1274 via pivoting of the elongated body 1722 about its connection point 1722a, to the first lower ring 1756. The distal portion of the elongated body 1772, which supports and engages the tissue piercing portion 1774, is configured with a deployment locking mechanism, such as a locking tab (not depicted, but similar to 1722b discussed above) that is adapted to maintain the securement element 1770 in the deployed position (not shown) by engaging the first upper ring 1754 and resisting radially inward movement of the tissue piercing portion 1774, after deployment. The securement elements 1770 may be deployed in a similar manner as the securement elements 1720 discussed above.

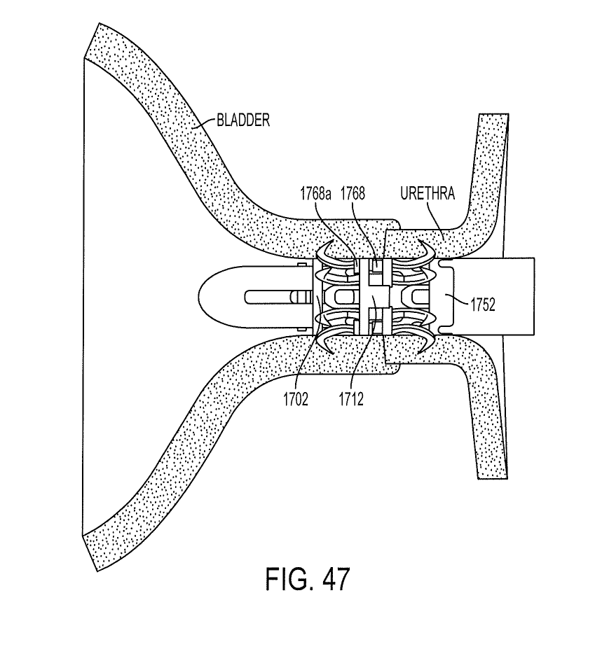

The first and second ring assemblies 1702, 1752 have corresponding alignment and retaining features adapted to facilitate measured, locking engagement of the ring assemblies 1702, 1752 during the performance of an anastomosis. First, with respect to alignment, the first lower ring 1706 contains alignment tabs 1712 adapted to engage corresponding alignment slots 1762 disposed on the second upper ring 1754. Engagement of the alignment tabs 1712 and alignment slots 1762 ensures that the first and second rings 1702, 1752 resist twisting with respect to each other, ensuring alignment of the locking mechanism and ensuring that tissue is pulled directly together. The locking mechanism of the first and second ring assemblies 1702, 1752 includes stepped tabs 1768 extending distally from the second upper ring 1754, which are sized and positioned to engage the first lower ring 1706 in the aperture 1718 formed by the leg portions 1708a of each strut assembly 1708. Each of the stepped tabs 1768 contains at least one, and preferably a plurality, of protrusions 1768a and corresponding recesses 1768b positioned for engagement with the first lower ring 1706 at varying intervals of proximity between the first and second ring assemblies 1702, 1752. That is, as depicted in FIG. 47, during deployment, a protrusion 1768a from the second ring assembly 1752 is received in a corresponding aperture 1718 of the first ring assembly 1702 such that a portion of the first lower ring 1706 is received within one of the recesses 1768b. If it is desired to bring the adjacent tissue portions to be joined closer together, the first and second ring assemblies, 1702, 1752, can be brought closer together thereby causing the second or lower protrusion 1768a on the stepped tabs 1768 to be received within the apertures 1718 of the first ring assembly 1702, locking the ring assemblies 1702, 1752 in place closer to each other. As depicted in FIGS. 45 and 46, and described above, the tissue engagement portions 1720, 1770 are disposed opposite each other such that, during deployment and joining of the ring assemblies 1702, 1752, the respective tissue engaged by each is drawn inwardly, forming an anastomosis.

The first and second ring assemblies may each be of unitary construction, or may be bonded or together using techniques discussed herein. One or more of the features disclosed herein with respect to the embodiments of FIGS. 45 and 46 may also be used in connection with the other embodiments disclosed herein. Likewise the embodiments of FIGS. 45 and 46 may incorporate features of other embodiments disclosed herein.

### Insertion Instrument

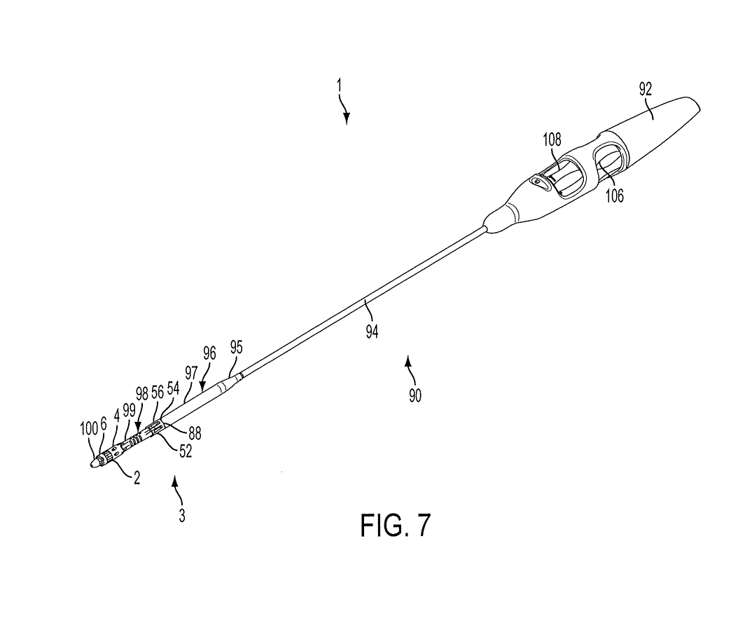

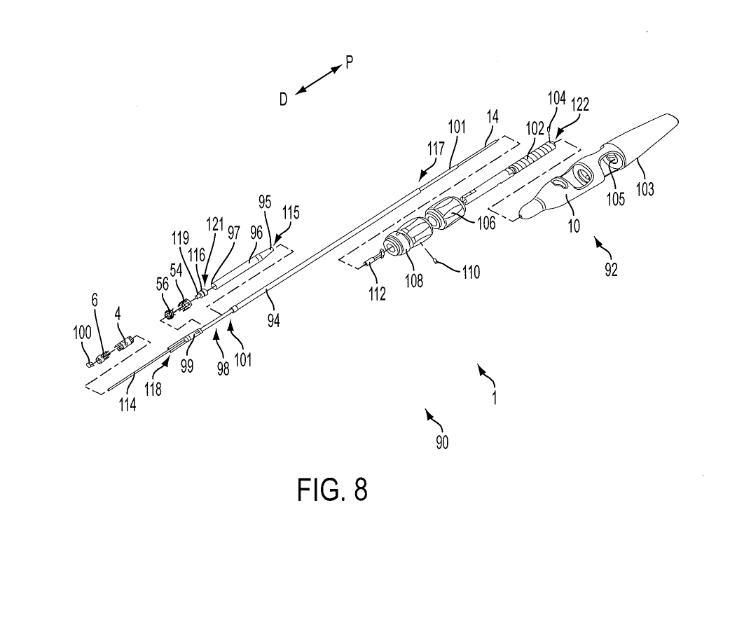

Turning now to FIGS. 7 and 8, an exemplary embodiment of an insertion instrument 90 is shown. The insertion instrument 90 may be used to (i) insert the second ring assembly 52 in a specific anastomosis site and the first ring assembly 2 into adjacent tissue, e.g. the bladder and urethra or other hollow body parts, (ii) separately deploy the respective securement elements 20, 62, and (iii) couple the second ring assembly 52 and the first ring assembly 2 together. The insertion instrument 90 can be withdrawn from the patient leaving the second ring assembly 52 and the first ring assembly 2 in place, sealing the anastomosis.

As shown in FIG. 7, the insertion instrument 90 includes a handle assembly 92, a tube 94 (which can be flexible or rigid but is preferably flexible), an outer housing 96, an implant support 98 and a deployer 100 located at the distal tip of the insertion instrument 90. The flexible tube 94 is a generally elongate tube. The outer housing 96 is tube-shaped with a flexible tube-engaging portion 95 that tapers into a circumference similar to that of the flexible tube 94 and a second collar mounting portion 97, having a circumference similar to that of the second collar 54. The implant support 98 defines a generally cylindrical distal implant mounting portion 99 and a generally elongate, tubular implant support shaft 101 extending proximally from the implant mounting portion 99 into the flexible tube 94 (seen best in FIG. 8). The deployer 100 is generally conical and is mounted distally on an elongate deployer shaft 114 (seen best in FIG. 8).

As shown in FIG. 7, when the insertion instrument 90 is assembled, the flexible tube 94 is disposed between the handle assembly 92 and the outer housing 96. The implant mounting portion 99 of the implant support 98 extends distally from the second collar mounting portion 97 of the outer housing 96. The deployer 100 extends distally from the implant mounting portion 99 of the implant support 98.

As best seen in FIG. 8, at least a portion of the flexible tube 94, implant support 98, and outer housing 96 respectively define lumens 117, 118 and 115 extending therethrough. The diameter of the lumen 117 within the flexible tube 94 and lumen 115 of the outer housing 96 are each sized to slideably receive a portion of the implant support shaft 101. Further, the diameter of the lumen 115 of the outer housing 96 is greater than the diameter of the implant mounting portion 99 of the implant support 98, such that the outer housing 96 can receive a portion of the implant mounting portion 99. The lumen 118 of the implant support 98 is sized to slideably receive a portion of the deployer shaft 114. Thus, when the implant support shaft 101 and deployer shaft 114 are received within the lumen 117 of the flexible tube 94, as the insertion instrument 90 is assembled, the flexible tube 94, implant support shaft 101, and deployer shaft 114 form coaxial elongate members. Due to this coaxial arrangement, the implant support shaft 101 and deployer shaft 114 can translate axially with respect to the handle assembly 92 within the lumens 117, 115 of the flexible tube 94 and outer housing 96.

Furthermore, as seen in FIG. 8, the implant support shaft 101 is of a length such that the implant mounting portion 99 can extend distally from the outer housing 96 while a portion of the implant support shaft 101 is received within the handle assembly 92 when the insertion instrument 90 is assembled. Similarly, the deployer shaft 114 is of a length such that the deployer 100 can extend distally from the implant mounting portion 99 when the insertion instrument 90 is assembled while a portion of the deployer shaft 114 is proximally received within the handle assembly 92.

As seen in FIG. 8, a urethra side cam 116, which defines a cone shape with a lumen 121 and a tapered portion 119, is slideably mounted in the second collar mounting portion 97 of the outer housing 96. The tapered portion 119 of the urethra side cam 116 extends distally from the second collar mounting portion 97 of the outer housing 96. The lumen 121 of the urethra side cam 116 is sized to slideably receive the implant support shaft 101 and is in coaxial alignment with the outer housing 96 (as seen best in FIG. 13C). Thus, as best seen in FIG. 13C, in the assembled insertion instrument 90, the implant support shaft 101 can pass through the lumen 121 of the urethra side cam 116.

As shown in FIG. 7, when the anastomosis system 1 is assembled, the first ring assembly 2 and second ring assembly 52 are mounted in spaced relation to each other, on the distal portion of the insertion instrument 90. The second collar 54 engages the second collar mounting portion 97 of the outer housing 96, via the instrument engaging elements 88. The second central ring 56 is mounted proximally on the implant mounting portion 99 of the implant support 98 and positioned distally of the second collar 54, with the second ring securement elements 62 extending axially within the second collar 54 and the outer housing 96 (also seen in FIG. 13C). As best seen in FIG. 13C, the tapered portion 119 of the urethra side cam 116 extends into the lumen 80 of the second collar 54 and engages the inner surfaces 70 of the second securement elements 62. The first collar 4 is mounted distally on implant mounting portion 99 of the implant support 98. The first central ring 6 is mounted on the deployer 100 and positioned proximal of the first collar 4.

The second ring assembly 52 and first ring assembly 2 are mounted on the insertion instrument 90 such that the first ring interconnecting elements 47 are axially aligned with the second ring interconnecting elements 84 and the second central ring locks 86 are axially aligned with the support surfaces 50 of the first collar 4. In the embodiment shown, the first and second ring assemblies 2, 52 are not intended to rotate about their common longitudinal axis during deployment of the securement elements 24, 62 and attachment to each other. The second ring securement element locking members 48 are also axially aligned with the second ring securement element cam surfaces 72.

As shown in FIGS. 7, 8 and 11, the handle assembly 92 includes an actuation shaft 102, a hollow grip member 103, a stopper cross-pin 104, a rotary actuation knob 106 and a rotary selection knob 108. The rotary selection knob 108 includes an opening defining a plunger pin receiver 109 that is sized to receive a plunger pin 110. The handle assembly 92 further includes an adapter 112 that is mechanically coupled to the actuation shaft 102.

In general, the handle assembly 92 is assembled such that the stopper cross pin 104, pin rotary actuation knob 106, rotary selection knob 108, plunger pin 110 and adapter 112 are mounted on or in the actuation shaft 102. Additionally, the actuation shaft 102, stopper cross pin 104, pin rotary actuation knob 106, rotary selection knob 108, plunger pin 110, adapter 112 are mounted within a lumen 105 extending within the hollow grip member 103.

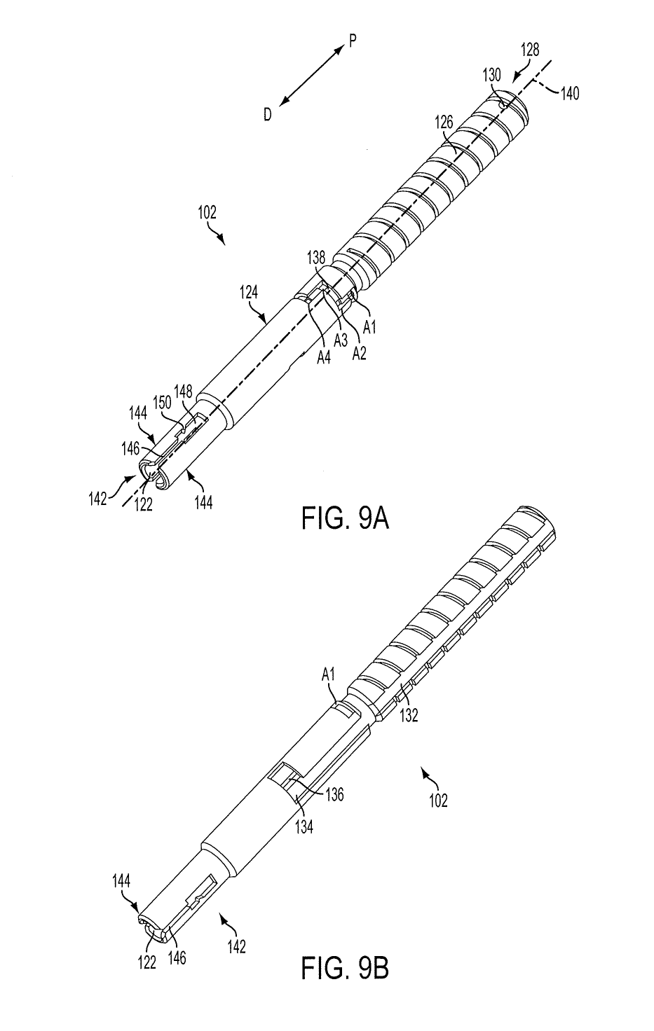

Turning now to FIGS. 9A and 9B, detailed views of the actuation shaft 102 and adapter 112 are shown. As pictured, the actuation shaft 102 has an internal lumen 122 defining a passageway through an elongated tubular body 124, with the passageway sized to receive a portion of the deployer shaft 114 and a portion of the adapter 112. When the insertion instrument 90 is assembled, the deployer shaft 114 is fixed within the lumen 122 of the actuation shaft 102 such that the axial or rotational motion of the actuation shaft 102 is transferred to the deployer shaft 114.

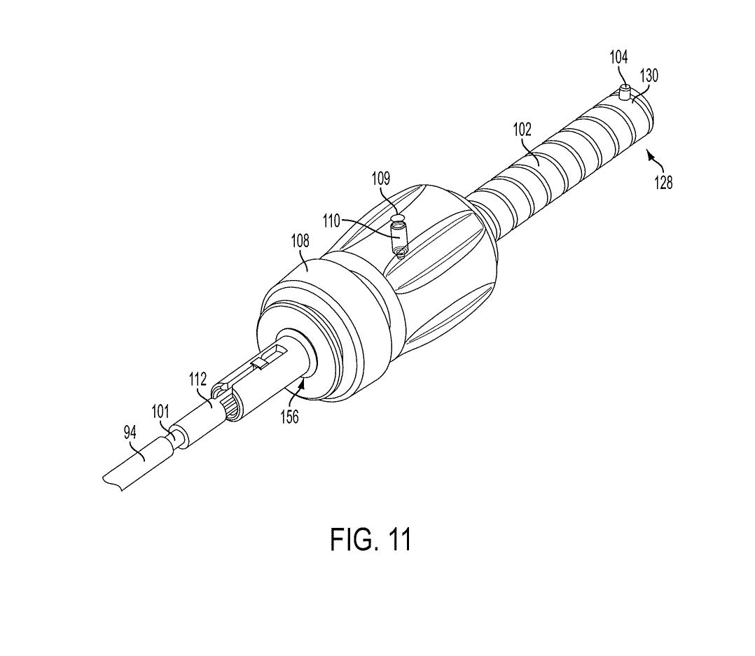

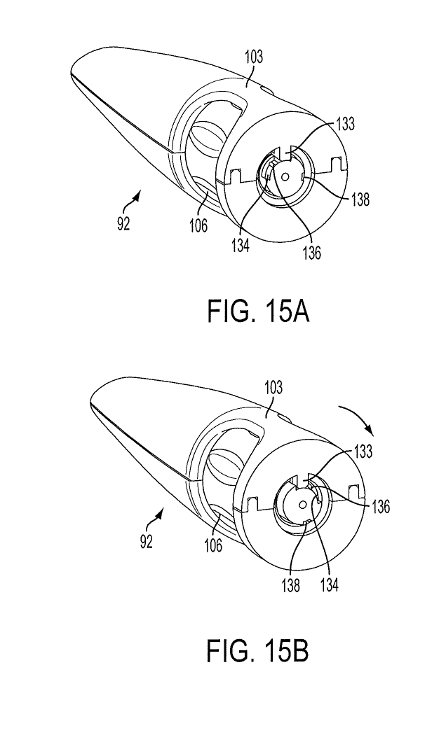

The outer surface of the tubular body 124 has a threaded portion 126 located adjacent the proximal end 128. The proximal end 128 of the actuation shaft 102 also defines a stopper cross-pin opening 130 for receiving the stopper cross-pin (as best seen in FIG. 11). Additionally, the actuation shaft 102 includes a device guide slot 132 extending distally from the proximal end 128 along the length of the threaded portion 126. The device guide slot 132 is sized to receive the hollow grip release detent 133 of the hollow grip member 103 (shown in FIGS. 15A and 15B) to permit axial sliding of the actuation shaft 102 with respect to the hollow grip member 103 during assembly and use of the insertion instrument 90. As shown in FIG. 9B, the device guide slot 132 terminates in a circumferential recess 134 that defines an outward extending actuation shaft detent 136. The actuation shaft detent 136 cooperates with the hollow grip release detent 133 of the hollow grip member 103 to provide an audible sound and physical indication that the insertion instrument 90 is set to the “Release” position (as best seen in FIGS. 15A and B).

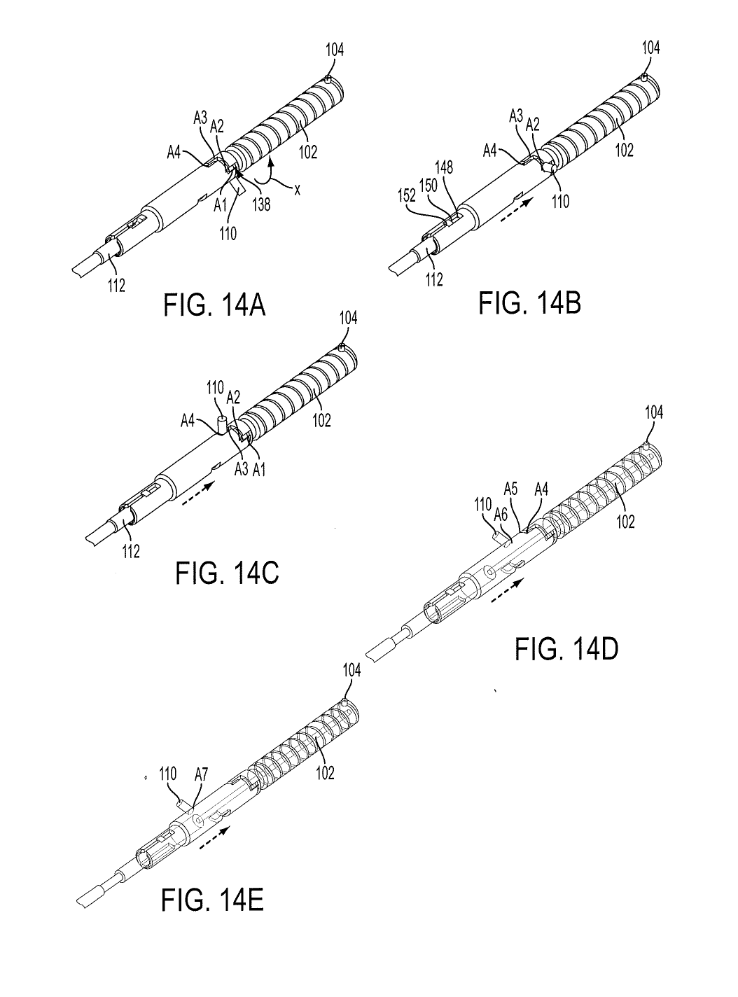

As best seen in FIG. 9A, the actuation shaft 102 further includes a plunger guide 138 that defines a grooved and angled pathway. The angled pathway of the plunger guide 138 defines a series of right angles A1-A4 traced by the plunger guide 138 alternating between either extending: (1) counterclock-wise and perpendicular to a longitudinal axis 140 of the actuation shaft 102 (preferably at 72°); or (2) distally and parallel to the longitudinal axis 140 of the actuation shaft 102. The plunger guide 138 has a width adapted to receive a portion of the plunger pin 110 when the insertion instrument 90 is assembled. As discussed below in detail with respect to FIGS. 14A-14E, movement of the plunger pin 110 through the plunger guide 138 allows the rotary selection knob 108 to select the second ring assembly 52 or first ring assembly 2 for deployment or coupling.

Still referring to FIGS. 9A and 9B, the distal portion 142 of actuation shaft 102 includes longitudinally extending arms 144, which define an axially extending adaptor slot 146. The adaptor slot 146 terminates in an adaptor guide receiver 148 defining an aperture with a protruding adaptor detent 150.

Although the embodiment of an actuation shaft 102 shown in FIGS. 9A and 9B is of unitary construction, one skilled in the art will appreciate that an actuation shaft may be an assembly of two or more separate shafts (not shown). An actuation shaft formed from separate shafts may advantageously permit the independent deployment of the ring assemblies 2, 52,

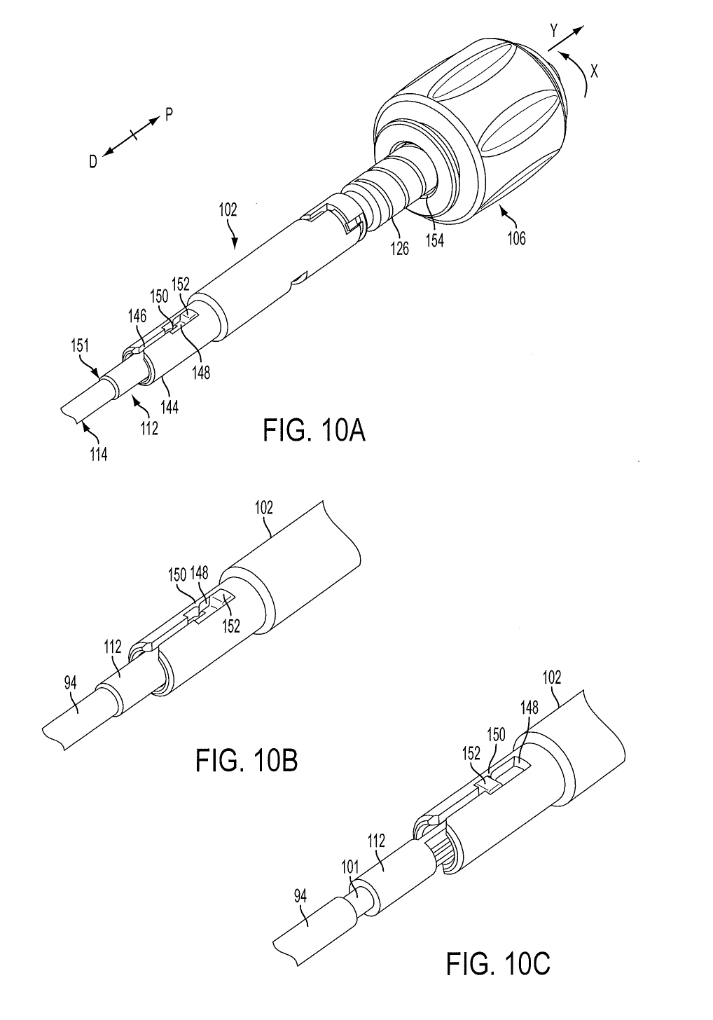

As seen in FIGS. 10A-10C, the adaptor 112 is generally tubular with a lumen 151 defining a passageway therethrough and has an outwardly extending adaptor guide 152. The lumen 151 is sized to slideably receive the deployer shaft 114 and a portion of the implant support shaft 101. Furthermore, the portion of the implant support shaft 101 received within the lumen 151 is fixed to the adaptor 112 to restrict axial and rotational motion of the adaptor 112 with respect to the implant support shaft 101,

The adaptor 112 may be inserted into the lumen 122 by spreading the longitudinally extending arms 144 apart to allow the adaptor guide 152 to move through the adaptor slot 146 and into the adaptor guide receiver 148 proximal of the adaptor detent 150. When the adapter 112 is received in the lumen 122 of the actuation shaft 102, the adaptor guide receiver 148 is free to move proximally with respect to the adaptor guide 152 until the first ring securement elements 20 of the first ring assembly 2 are deployed. As shown in FIG. 10C, after the proximal translation of the actuation shaft 102 and adaptor guide receiver 148, the adaptor detent 150 engages the adaptor guide 152 to restrict both longitudinal and rotational motion of the adaptor 112 with respect to the actuation shaft 102. Thus, when the adaptor guide receiver 148 is engaged by the adaptor detent 150 (i.e., after deployment of the first ring assembly 2), axial translation of the actuation shaft 102 will carry the adaptor 112 (and the implant support shaft 101 mounted thereto) in a coordinating movement.

Additionally, as seen in FIG. 10A, the threaded portion 126 of the actuation shaft 102 passes through the rotary actuation knob 106. The rotary actuation knob 106 is provided with a threaded lumen 154 that matingly engages the threaded portion 126 of the actuation shaft 102. Thus, rotation of the rotary actuation knob 106 in the counter-clockwise direction with respect to the actuation shaft 102 causes the actuation shaft 102 to translate proximally with respect to the rotary actuation knob 106 (as shown by arrows x and y in FIG. 10A). Likewise, rotation of the rotary actuation knob 106 in the clockwise direction with respect to actuation shaft 102 causes the actuation shaft 102 to translate distally with respect to the rotary actuation knob 106.

Turning now to FIG. 11, the ring-shaped rotary selection knob 108 is shown mounted on the actuation shaft 102 with the plunger guide 138 (not shown) passing through a lumen 156 of the rotary selection knob 108. The plunger pin 110 is shown mounted in the plunger pin receiver 109 of the rotary selection knob 108 with a portion of the plunger pin 110 extending into the lumen 156 of the rotary selection knob 108. Thus, when the insertion instrument 90 is assembled, the plunger pin 110 engages the plunger guide 138 of the actuation shaft 102 and is moved laterally by rotation of the rotary selection knob 108 with respect to the actuation shaft 102. The longitudinally extending portions of the plunger guide 138 permit axial translation of the actuation shaft 102 with respect to the plunger pin 110 and rotary selection knob 108. Also, the rotary selection knob 108 can include labels or markings positioned to indicate the selected operation selected by the rotary selection knob 108 (i.e., Locked, Bladder, Urethra, Anastomosis, and Release).

Additionally, as shown in FIG. 11, the stopper cross-pin 104 is mounted within the stopper cross-pin opening 130 at the proximal end 128 of the actuation shaft 102. The stopper cross-pin 104 is adapted to restrict axial translation of the proximal end 128 of the actuation shaft 102 with respect to the hollow grip member 103 in a distal direction past the rotary actuation knob 106.

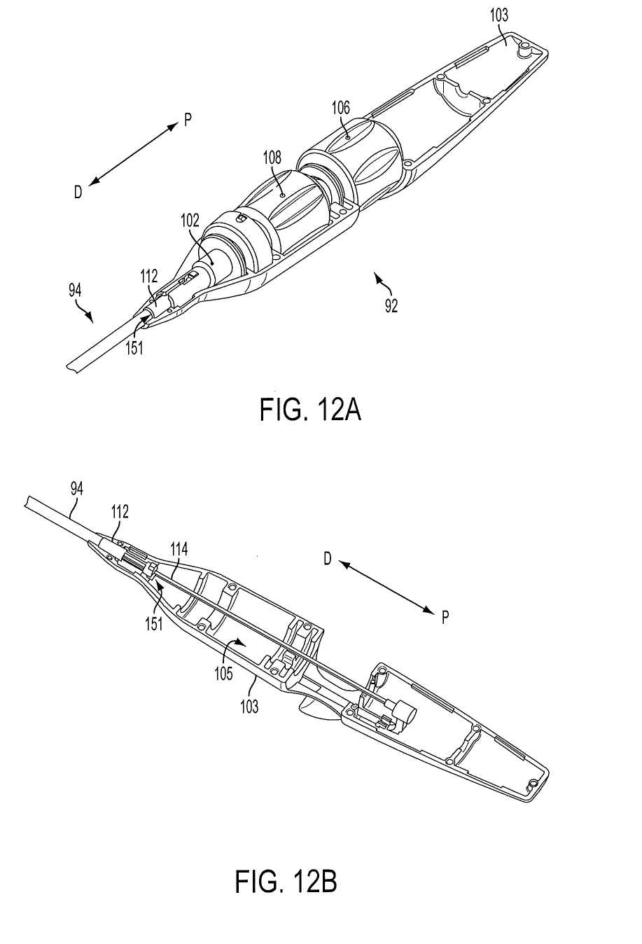

Referring now to FIGS. 12A and 12B, an example of a partially assembled handle assembly 92 is shown. In FIG. 12A, the rotary selection knob 108 and rotary actuation knob 106 are shown both mounted on the actuation shaft 102, with the rotary selection knob 108 being mounted proximally of the rotary actuation knob 106. As shown here, in the initial or “Locked” position, the adaptor 112 extends distally from the actuation shaft 102 and abuts the flexible body 94 which is fixed to the hollow grip member 103. The actuation shaft 102 with knobs 106, 108 are disposed within the hollow grip member 103.

In FIG. 12B, the handle assembly 92 is shown with only the deployer shaft 114 and adapter 112 mounted within the hollow grip member 103. As shown, the deployer shaft 114 extends through the hollow grip member 103 while the deployer shaft 114 passes through the lumen 151 of the adaptor 112, and would likewise pass through the lumen 122 of the actuation shaft 102 if the actuation shaft 102 were shown positioned in the hollow grip member 103.

Turning now to FIGS. 13A to 13B, detail of the implant mounting portion 99 of the implant support 98 is shown. The implant mounting portion 99 is generally cylindrical and comprises a first ring mounting portion 160 and a second ring mounting portion 162.

The first ring mounting portion 160 includes at least one axially extending first collar support member 164 and at least one axially extending and resiliently flexible first collar locking member 166. As seen best in FIG. 13B, the first collar 4 of the first ring assembly 2 is mountable on the first collar support member 164, with the first collar locking member 166 engaging the support surface 50 of the first collar 4. Thus, when the first collar locking member 166 axially extends and engages the support surface 50 of the first collar 4, as shown, the first collar locking member 166 restricts movement of the first collar 4 with respect to the implant support 98. However, a radially inward force applied to the first collar locking members 166 can cause the first collar locking members 166 to become disengaged from the first collar 4. When the first collar locking members 166 are disengaged from the first collar 4, the implant support 98 can slide through lumens 60 and 80 of the second central ring 56 and second collar 54 (see FIG. 6), such as during withdrawal of the insertion instrument 90.

As shown in FIG. 13C, the first central ring 6 is releasably retained on the deployer 100 of the insertion instrument 90 by protrusion of the deployer detent 113 into the circumferentially extending deployment slot 18 of the first central ring 6. As shown, the first central ring 6 is positioned distally with respect to the first collar 4, and the first ring securement elements 20 extend axially to a position within the outer circumference of the first collar 4.

Referring now to FIGS. 13A-13C, the second ring mounting portion 162 includes at least one flexibly resilient axially extending second ring support member 168 having proximally positioned a second ring undeployer cam 170 and a second ring deployer cam 171 positioned distally thereto. As best seen in FIG. 13B, the second ring undeployer cam 170 and the second ring deployer cam 171 are configured so that the second ring assembly wall 58 between the second ring securement element mounting members 64 of the second central ring 56 can be mounted on the second ring support members 168 between the second ring undeployer cam 170 and a second ring deployer cam 171. Thus, when the second ring support members 168 axially extend and the second central ring 56 is mounted thereon, the second ring undeployer cam 170 and a second ring deployer cam 171 restrict translation of the second central ring 56 with respect to the implant support 98. However, an inward force applied to the second ring support member 168 can cause the second ring support member 168 to become disengaged from the second central ring 56, thus allowing the implant support 98 to slide through lumens 60 and 80 of the second central ring 56 and second collar 54.

The second ring mounting portion 162 also includes at least one second ring securement element engaging cam member 163 extending axially from the implant mounting portion 99 of the implant support 98. The second ring securement element engaging cam members 163 are positioned between the second ring support members 168, about the circumference of the implant mounting portion. The second central ring 56 may be mounted on the second ring mounting portion 162 such that the second ring securement element engaging cam members 163 are positioned distally of and directed towards the second ring securement element cam surfaces 72.

Referring now to FIGS. 14A-14E, the movement of the actuation shaft 102 relative to the hollow grip member 103, during operation of the insertion instrument 90, is illustrated. As shown in FIG. 14A, in the initial or “Locked” position, the plunger pin 110 is received in the proximal portion of the plunger pin guide 138. To allow deployment of the first ring assembly 2, the rotary selection knob 108 (as seen in FIG. 12A) is rotated counter-clockwise (shown by the arrow x in FIG. 14A) to slide the plunger pin 110 through the plunger guide 138. From the “Locked” deployment position, counter clockwise rotation of the rotary selection knob 108 causes the plunger pin 110 to move within the plunger pin guide 138 to angle A1, thereby selecting the “Bladder” deployment position.

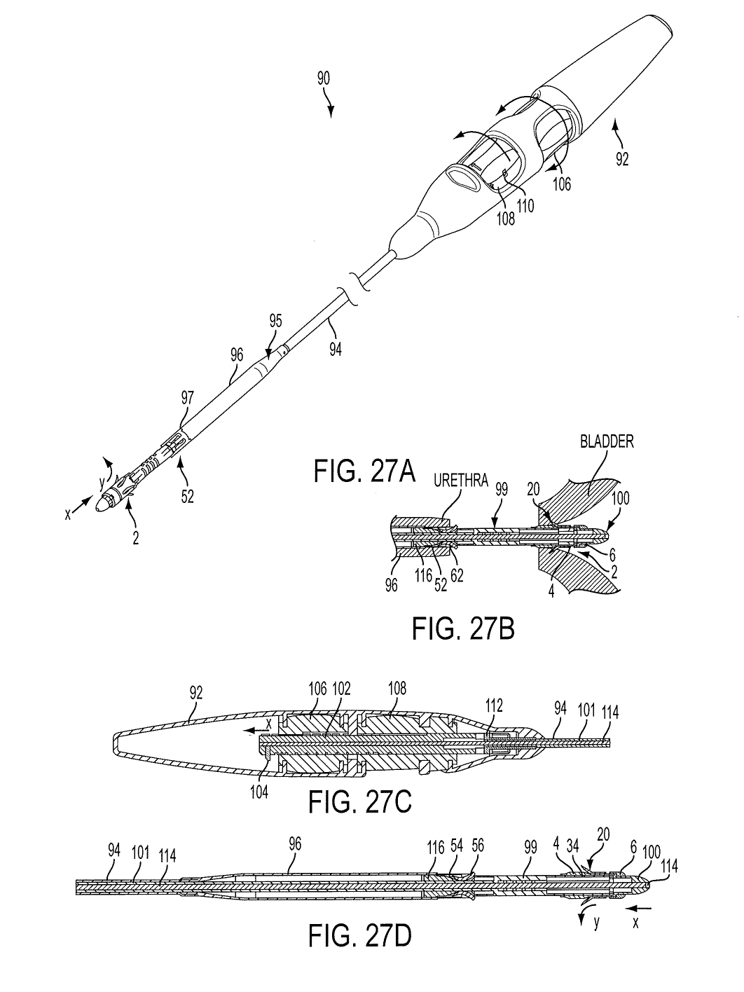

As shown in FIG. 14B, when the rotary selection knob 108 is in the “Bladder” deployment position, the insertion instrument 90 can deploy and undeploy the first ring assembly 2 to cause the first ring securement elements 20 to engage the surrounding tissue (i.e., bladder neck or other hollow body part). In the “Bladder” deployment position, the first ring assembly 2 can be deployed by proximal retraction of the actuator shaft 102 with respect to the hollow grip member 103 (not shown) and adapter 112, as shown by the arrow in FIG. 14B. Proximal retraction of the actuator shaft 102 can be effected by rotating the rotary actuation knob 106 (not shown) counter clockwise with respect to the actuator shaft 102, such that the threaded lumen 154 of the rotary actuation knob 106 engages the threaded portion 126 of the actuation shaft 102. As shown, engagement of the threaded portion 126 of the actuation shaft 102 during rotation of the rotary actuation knob 106 causes the actuation shaft 102 to move proximally such that the plunger guide 138 moves proximally about the plunger pin 110 and the position of the plunger pin 110 changes from A1 to A2. Because the deployer shaft 114 is fixed in lumen 122 of the actuation shaft 102, proximal translation of the actuation shaft 102 with respect to the hollow grip member 103 causes the deployer 100 to proximally retract with respect to the first collar 4, thereby deploying the first ring assembly 2 to engage the bladder or other tissue (discussed in detail below with respect to FIGS. 27A-27D).

Furthermore, as illustrated in FIG. 14B, proximal retraction of the actuation shaft 102 with respect to the adaptor 112 results in the adaptor guide receiver 148 to translate proximally about the adaptor guide 152 and causes the adaptor guide 152 to be engaged by the adaptor detent 150. Thus, with the actuator shaft 102 engaging the adaptor 112, further proximal translation of the actuation shaft 102 will carry the adaptor 112 in a coordinating motion.

Referring now to FIG. 14C, to select the insertion instrument 90 for partial deployment of the second ring assembly 52, the rotary selection knob 108 may be turned counterclockwise to carry the plunger pin 110 to position A3 of the plunger guide 138. When the plunger pin 110 is in position A3 of the plunger guide 138, the insertion instrument is in the “Urethra” deployment position. As shown in FIG. 14C, the rotary actuation knob 106 (not shown) can then be rotated counter clockwise with respect to the actuator shaft 102 to cause proximal retraction of the actuation shaft 102 with respect to the hollow grip member 103 such that the plunger pin guide 138 moves about the plunger pin 110 and the plunger pin 110 position changes from position A3 to A4.

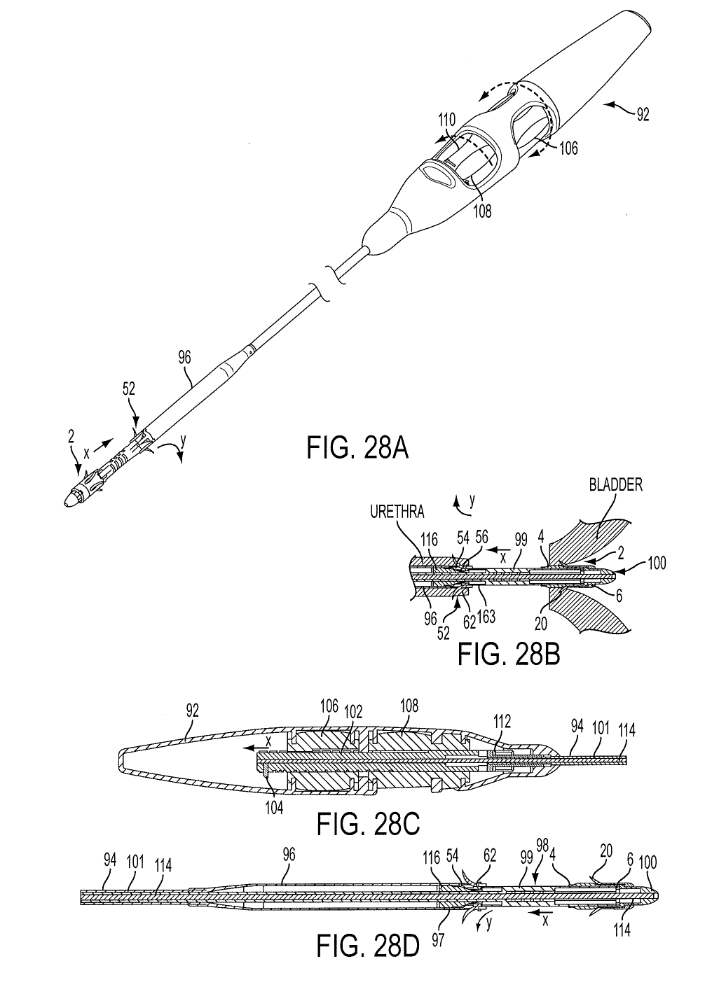

Because the implant support shaft 101 is mounted on the adaptor 112, which is engaged by the actuation shaft 102 in the Urethra position, proximal retraction of the actuation shaft 102 results in proximal translation of the implant support 98 with respect to the hollow grip member 103 and outer housing 96. This proximal translation of the implant support 98, with respect to the hollow grip member 103 and outer housing 96, results in partial deployment of the second ring assembly 52 (discussed in detail below with respect to FIGS. 28A-28D).

As shown in FIG. 14D, to select the insertion instrument 90 for full deployment of the second ring assembly 52, the rotary selection knob 108 (not shown) may again be turned counterclockwise with respect to the actuation shaft 102, thereby carrying the plunger pin 110 to position A5. When the plunger pin 110 is in position A5 of the plunger guide 138, the insertion instrument 90 is in the “Anastomosis” position. The rotary actuation knob 106 (not shown) can then be rotated counter clockwise with respect to the actuator shaft 102 to again cause proximal retraction of the actuation shaft 102 with respect to the hollow grip member 103 (not shown). Retraction of the actuation shaft 102 with respect to the hollow grip member 103 when the plunger pin 110 is in position A5 shifts the position of the plunger pin 110 from A5 to A6 within the plunger guide 138. When the plunger pin 110 moves from A5 to A6 by proximal retraction of the actuation shaft 102 with respect to the hollow grip member 103, the result is further proximal translation of the implant support 98 with respect to the handle assembly 92 and outer housing 96. This further proximal translation of the implant support 98 results in full deployment of the second ring assembly 52 (discussed in detail below with respect to FIGS. 29A-29D).

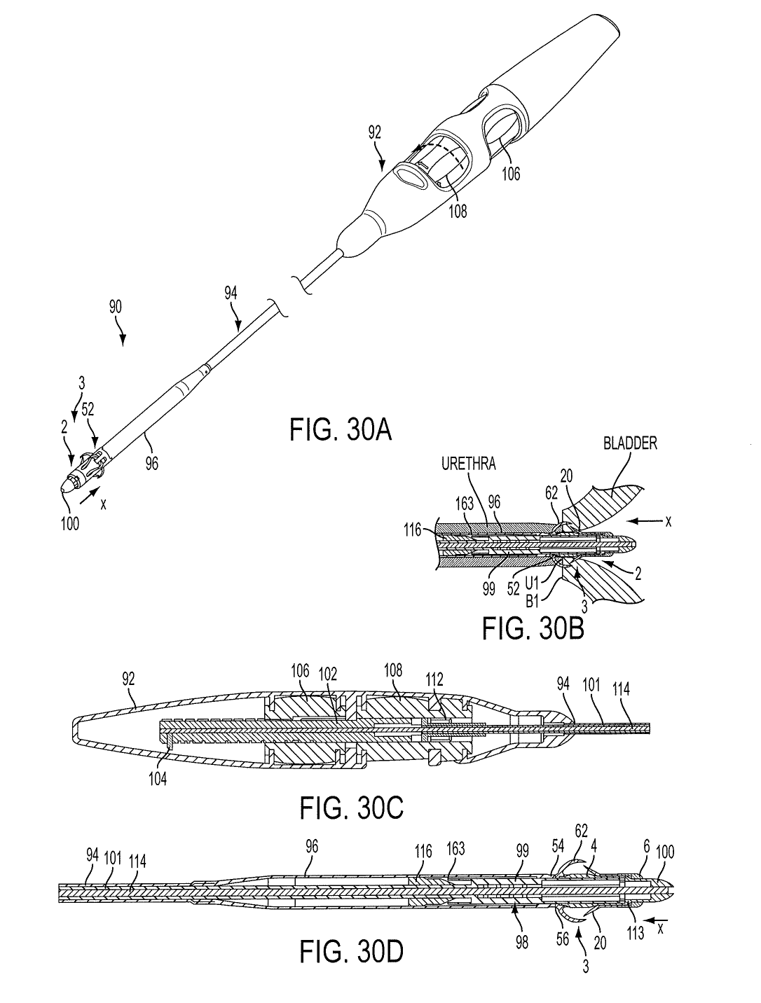

As shown in FIG. 14E, approximation of the anastomosis can be achieved by further counter clockwise rotation of the rotary actuation knob 106 with respect to the actuator shaft 102 when the insertion instrument 90 is in the “Anastomosis” position. When the plunger pin 110 rests in position A6, rotation of the rotary actuation knob 106 with respect to the actuator shaft 102 causes the actuation shaft 102 to translate proximally with respect the handle assembly, thereby causing the plunger pin guide 138 to move around the plunger pin 110 until the plunger pin 110 is in position A7. Proximal translation of the actuation shaft 102 with respect to the hollow grip member 103 draws the first ring assembly 2 towards the second ring assembly 52 (discussed in detail below with respect to FIGS. 30A-30D). Furthermore, when the first ring assembly 2 and second ring assembly 52 are deployed and secured to the surrounding tissue (e.g., bladder and urethra, respectively), approximation of the first ring assembly 2 towards the second ring assembly 52 draws the hollow body parts, such as bladder and urethra tissue, towards anastomosis. Interconnecting engagement of the first ring assembly 2 and second ring assembly 52 secures the anastomosis.

Turning now to FIGS. 15A and 15B, a cross-section of the handle assembly 92 is shown to illustrate structures cooperating during the release of the first ring assembly 2 and second ring assembly 52 from the insertion instrument 90. As shown, the hollow grip member 103 includes hollow grip release detent 133, which extends into lumen 105 of the handle assembly 92. When the insertion instrument 90 is assembled, the hollow grip release detent 133 is disposed within the device guide slot 132 (not shown) and circumferentially extending recess 134 (as best seen in FIG. 9B). FIG. 15A shows the relative position of the hollow grip release detent 133 within the circumferentially extending recess 134 during insertion of the insertion instrument 90 and deployment and coupling of the second and first ring assemblies 52, 2 (i.e., the initial position, “Bladder” position, “Urethra” position, and “Anastomosis” position). FIG. 15B shows the relative position of the hollow grip release detent 133 within the circumferentially extending recess 134 during release of the second and first ring assemblies 52, 2 from the insertion instrument 90 (i.e. the “Release” position) and withdrawal of the insertion instrument 90 from the body.

As can be seen from FIGS. 15A and 15B, the second and first ring assemblies 52, 2 (not shown) can be released from the insertion instrument 90 subsequent to coupling to the second and first ring assemblies 52, 2 by rotation of the rotary selection knob 108 to the “Release” position past the actuation shaft detent 136. The engagement of the hollow grip release detent 133 with the actuation shaft detent 136 provides an audible and physically perceptible indication that the insertion instrument 90 (not shown) in the “Release” position. Furthermore, because the deployer shaft 114 (not shown) is fixed to the actuation shaft 102 (not shown), rotation of actuation shaft 102 results in coordinating motion of the deployer 100 (not shown). Rotation of the deployer 100 causes the deployer detent 113 and the deployer 100 to rotate within circumferentially extending deployment slot 18 of the first central ring 6 and into device release groove 16 (shown in FIG. 2). When the deployer detent 113 the deployer 100 is positioned in the device release groove 16 (not shown) of the first central ring 6 (not shown), the deployer 100 can slide through the lumen 10 of the first central ring 6. Furthermore, in the Release position, the deployer 100 and implant mounting portion 99 of the implant support 98 (not shown) can slide through the lumens 35, 60, 80 (not shown) of the first collar 4, second central ring 56 and second collar 54 (not shown).

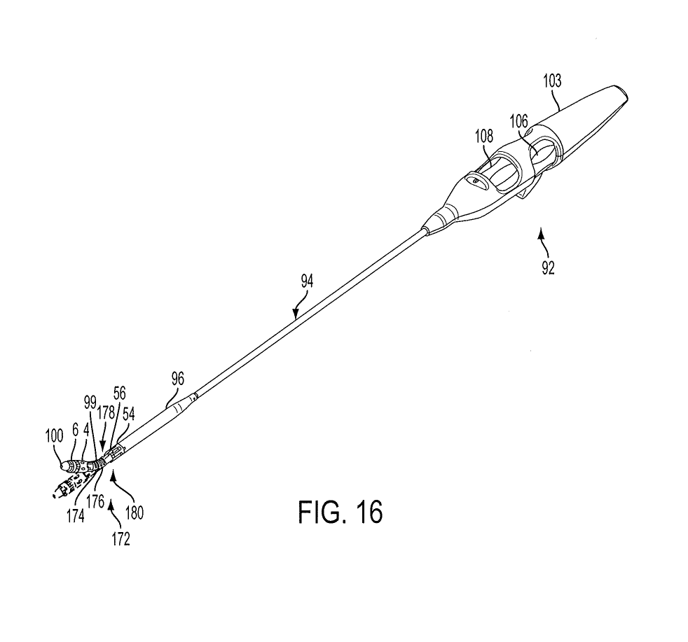

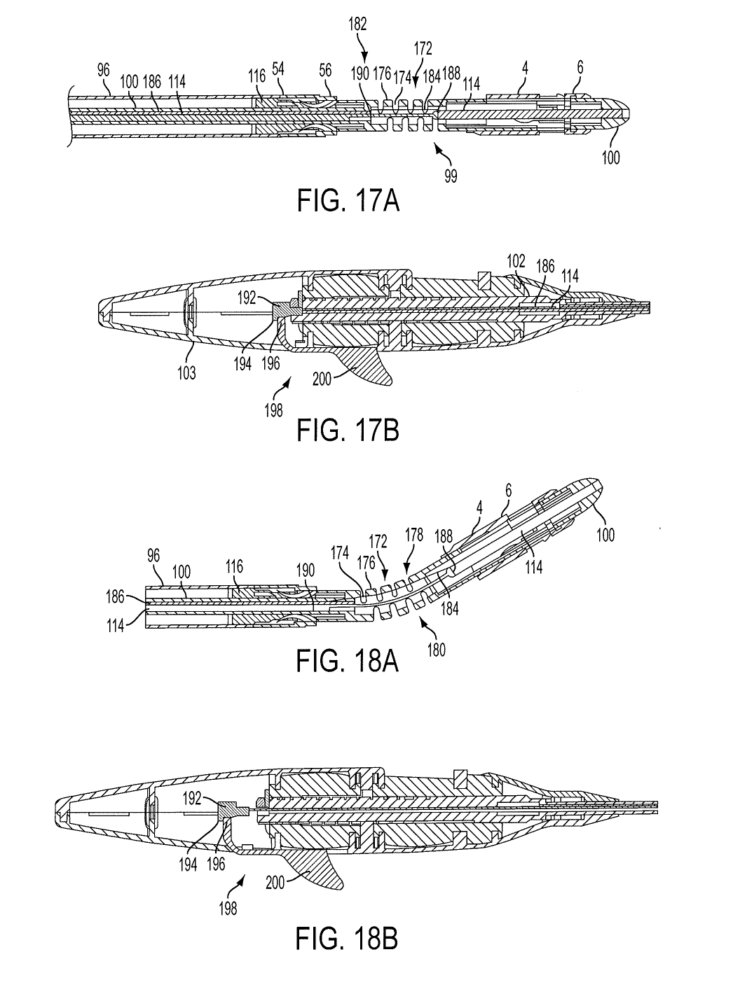

Turning now to FIGS. 16-20, the insertion instrument 90 has flexible portions that allow manipulation of the insertion instrument 90, to adjust to the natural curvature of a patient's anatomical structures. As shown in FIG. 16, the insertion instrument 90 includes an optional shaft flexing portion 172 (also seen in FIGS. 17A and 18A). The shaft flexing portion 172 is defines a plurality of slits 174 defining a plurality of circumferential wall supports 176. As shown in FIG. 18A, the slits 174 define open areas within the implant mounting portion 99 of the implant support that, due to the absence of material, allow the wall supports 176 on the inner side 178 to converge and on the outer side 180 to spread further apart, thereby bending the shaft flexing portion 172.

Turning now to FIGS. 17A and 17B, the flexing assembly 182 on the insertion instrument 90, which provides for flexing of the shaft flexing portion 172 is shown. The flexing assembly 182 includes a control cable 184, which is mounted distally of a tension shaft 186 within the insertion instrument 90. The control cable 184 is an elastic flexible cable having a first end 188 and a second end 190. The tension shaft 186 is a resilient elongated member sized to slide through the lumen 118 of the implant support shaft 101 while the deployer shaft 114 is also passing through the implant support shaft 101. The tension shaft 186 has a length such that a portion extends proximally from the actuation shaft 102 and a portion extends into the implant mounting portion 99 of the implant support 98.

As seen best in FIG. 17B, the tension shaft 186 extends proximally through the actuation shaft 102 and is fixed to a trigger engaging member 192. The trigger engaging member 192 is sized so that it cannot pass through the actuation shaft and includes a trigger engaging lip 194. The trigger lip 194 is adapted to engage the trigger extension 196 of trigger 198, such that axial proximal translation of the trigger 198 with respect to the hollow grip member 103 carries the trigger engaging member 192 and the tension shaft 186 axially in a coordinating proximal movement.

The trigger 198 includes a finger pull 200 extending radially outward from the hollow grip member 103. The trigger engaging member 192 member can be proximally translated by pulling the finger pull 200 of the trigger 198 proximally with respect to the hollow grip member 103.

As best seen in FIGS. 17A and 17B, the first end 188 of the control cable 184 is fixed to deployer shaft 114, distally of the shaft flexing portion 172 of the implant support 98. The second end 190 of the control cable 184 is fixed to the tension shaft 186, proximally of the shaft flexing portion 172.

Turning now to FIGS. 18A and 18B, proximal pressure applied to the finger pull 200 carries the trigger 198 proximally, with respect to the hollow grip member 103. The proximal translation of the trigger 198 carries the trigger extension 196 proximally into engagement with the trigger lip 194, thus urging the trigger engaging member 192, and tension shaft 186 proximally with respect to the actuation shaft 102. Proximal translation of tension shaft 186 through lumen 122 of the actuation shaft 102 results in tension being applied to the control cable 184. Applied tension causes the deployer shaft 114 attached to the first end 188 of the control cable 184 to flex. As shown in FIG. 18A, the flexing of the deployer shaft 114 causes the shaft flexing portion 172 of the implant support 98 to flex as well. Tension due to the elasticity of the flexing assembly 182 urges the flexing portion 172 to straighten upon release of pressure on the finger pull 200. As will be readily apparent to those skilled in the art, other means may also be used to effectuate directional movement of the deployer 100.

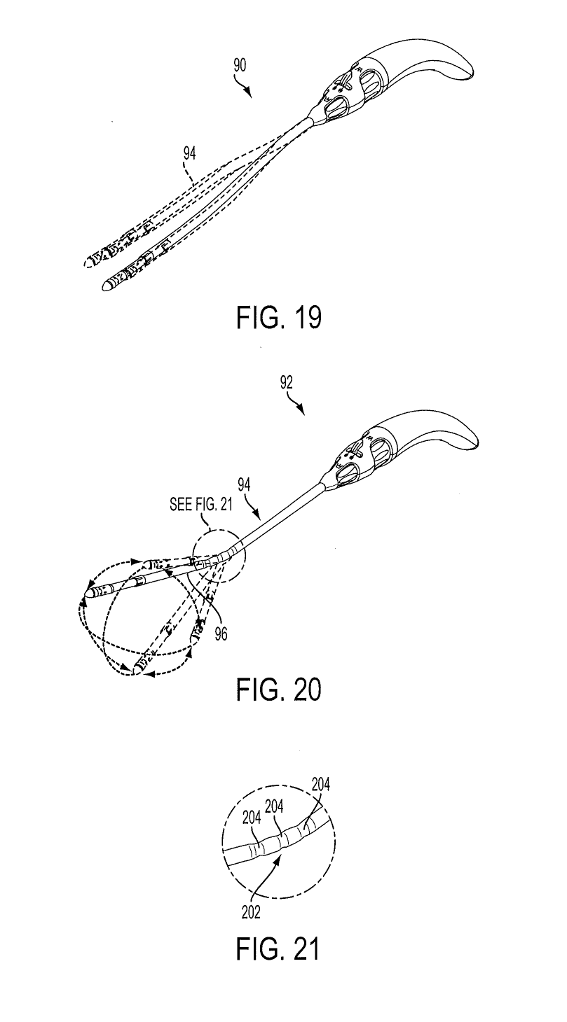

Turning now to FIG. 19, the insertion instrument 90 also has passive flexibility to allow further conformance to anatomical features. The flexible tube 94, implant support shaft 101, and deployer shaft 114 (located internally of flexible tube 94) are formed of resilient flexible material such that the insertion instrument can flex and bend to yield to resistance encountered during insertion of the insertion instrument 90 into curved anatomical structures.

As shown in FIGS. 20 and 21, in one embodiment, the insertion instrument 90 may include an optional shaft flexing portion 202 positioned proximally of the outer housing 96. The positioning of the flexing portion 202 permits 360° motion of the insertion instrument 90 extending distally from the junction between the flexible tube 94 and outer housing 96 and may be operated similarly to the flexing assembly 182 discussed in FIGS. 16 to 18. The shaft flexing portion is formed of circumferential grooves 204, which decrease the thickness of the outer housing 96, thereby concentrating flexibility in a similar manner to the flexing assembly 182.

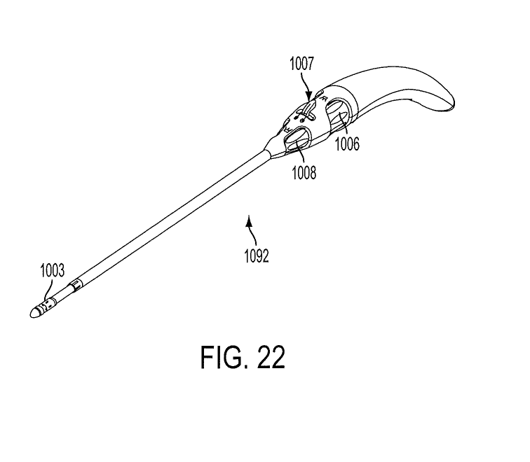

Turning now to FIG. 22, an alternate embodiment of a handle assembly 1092 is shown. The alternate handle assembly 1092 is provided with a device release switch 1007. Thus, rather than releasing the ring assembly 1003 from the insertion instrument 1092 by operation of the rotary selection knob 1008, as discussed with the knob 108, the ring assembly 1003 is released by depression of the device release switch 1007.

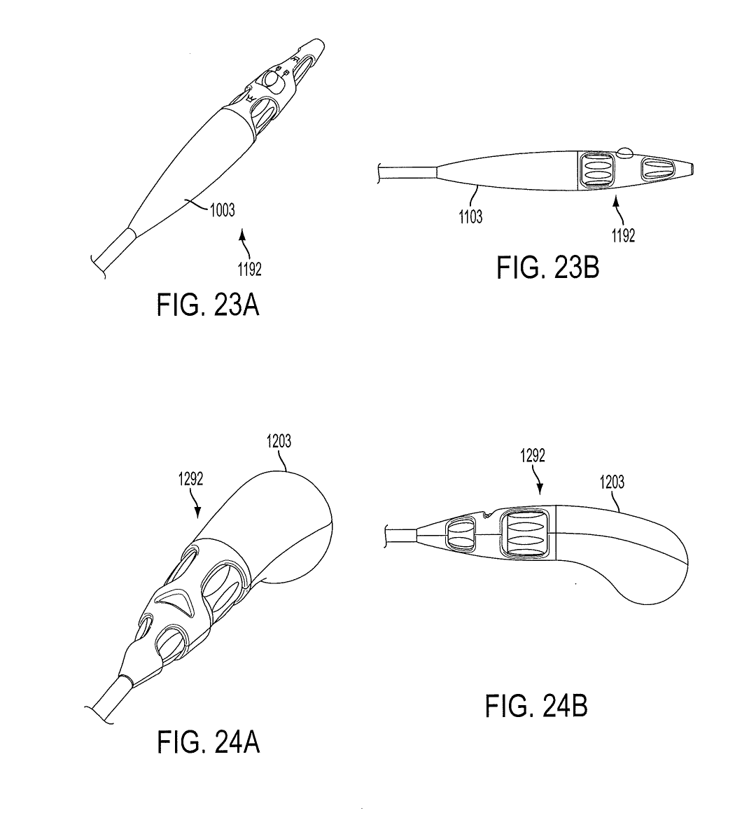

In FIGS. 22A and 22B and FIGS. 24A and 24B, alternate shapes of the hollow grip member 1103/1203 are shown. As shown in FIGS. 23A and 23B, the hollow grip member 1103 is a straight symmetrical shape. In contrast, as shown in FIGS. 24A and 24B, the hollow grip member 1103 is spherical. Alternate shapes suitable for comfortably gripping the hollow grip member 1103/1203 of the handle assembly 1192/1292 are also contemplated.

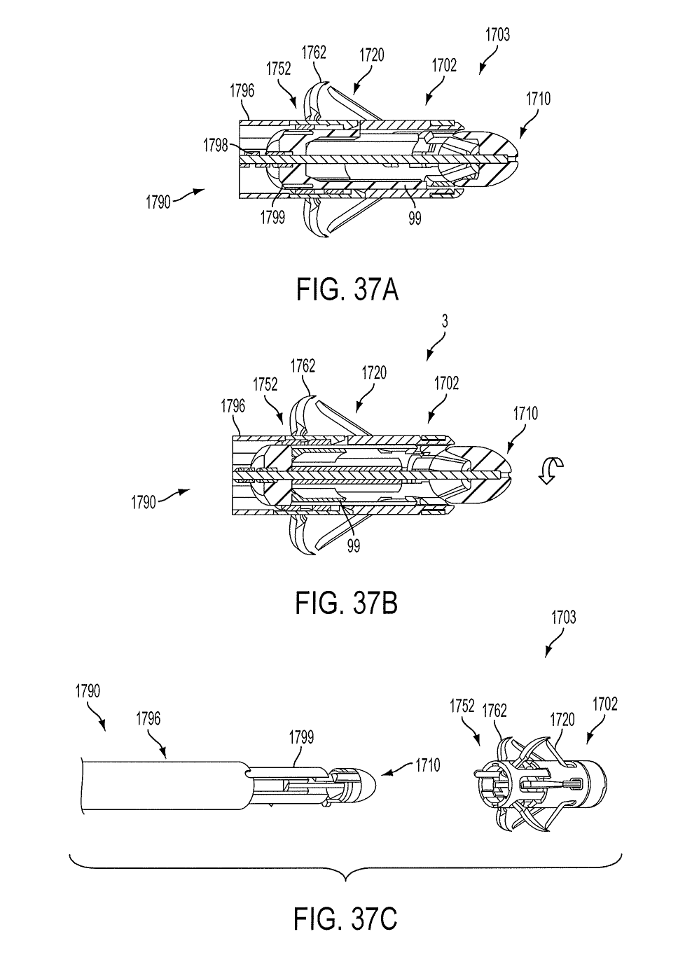

One skilled in the art will appreciate that alternate embodiments may incorporate different structures or designs for release of the ring assembly. One example of an alternate embodiment of a design for releasing the ring assembly 1703 from the insertion instrument 1790 is shown in FIGS. 37A-37C. As shown in FIG. 37A, the second ring assembly 1752 is mounted on the implant mounting portion 1799 of the implant support 1798 and the first ring assembly 1702 is mounted on the deployer 1710 during anastomosis. As shown in FIG. 37B, when the insertion instrument 1790 is operated to release the ring assembly 1703, the implant mounting portion 1799 of the implant support 1798 and the deployer 1710 are simultaneously rotated counter-clockwise with respect to the outer housing 1796 and the ring assembly 1703, as shown by the arrow in FIG. 37B. Rotation of the implant mounting portion 1799 and deployer 1710 with respect to the ring assembly 1703 disengages the ring assembly 1703 from the insertion instrument 1790, thereby allowing proximal translation of the insertion instrument 1790 away from the ring assembly 1703. As shown in FIG. 37C, translation or movement of the insertion instrument 1790 away from the ring assembly 1703 subsequent to disengagement of the ring assembly 1703 results in withdrawal of the insertion instrument 1790 from the patient and leaves the ring assembly 1703 in place holding anastomosis.

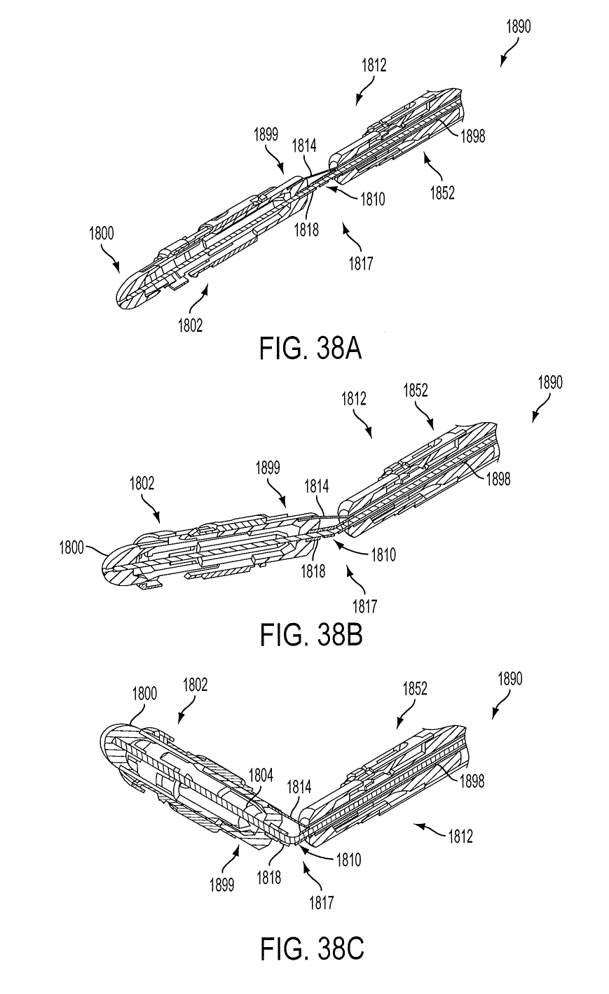

Additionally, one skilled in the art will appreciate that alternate designs for achieving flexibility or manipulability of an insertion instrument are possible, such as the embodiment of an insertion instrument 1890 depicted in FIGS. 38A to 38C. As shown in FIG. 38A, the insertion instrument 1890 includes a shaft flexing portion 1817 defined by the implant support shaft 1810. The shaft flexing portion 1817 defines a flexible tube having a plurality of segments 1818. As shown in FIG. 38A, the segments 1818 define open areas within the implant support shaft 1810 that, due to the absence of material, allow convergence towards or divergence from adjacent segments 1818, thereby allowing bending of the shaft flexing portion 1817.

Still referring to FIG. 38A, the flexing assembly 1812 of the insertion instrument 1890 includes a control wire 1814, which is mounted to the implant mounting portion 1899 of the implant support 1898 and to a trigger mechanism (not shown) on the handle portion of the insertion instrument 1890. The trigger mechanism (not shown) can be operated to apply tension to the control wire 1814, thereby causing the shaft flexing portion 1817 of the implant support shaft 1810 to bend or flex.

### Implantation Method

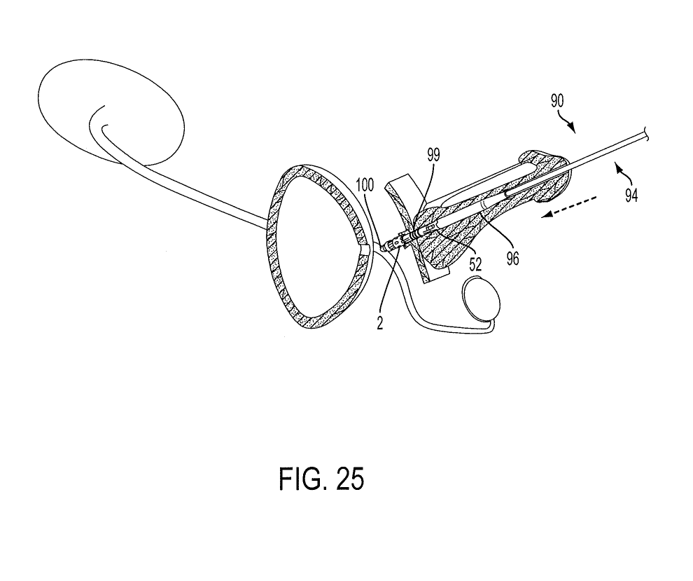

Referring to FIGS. 25-32, an exemplary method of using an insertion instrument 90 to create anastomosis of two vessels is shown. Although many types of anastomoses are possible using the device disclosed herein, an exemplary anastomosis of a bladder and urethra, such as one that may occur following removal of the prostate, is shown. While these figures depict the anastomosis of a bladder and urethra, the same or similar techniques should be understood as applying to the anastomosis of any other hollow organs or vesicles, such as blood vessels or intestines. Access to the anastomosis site may be achieved using natural orifices, such as the urethra as shown in FIGS. 25-32, suprapubicly, through incision or any other access port or via surgical means. As will be recognized by those skilled in the art, the specific insertion means will be determined by the type of anastomosis being performed and the available access areas in the specific body location where such anastomosis is being performed.

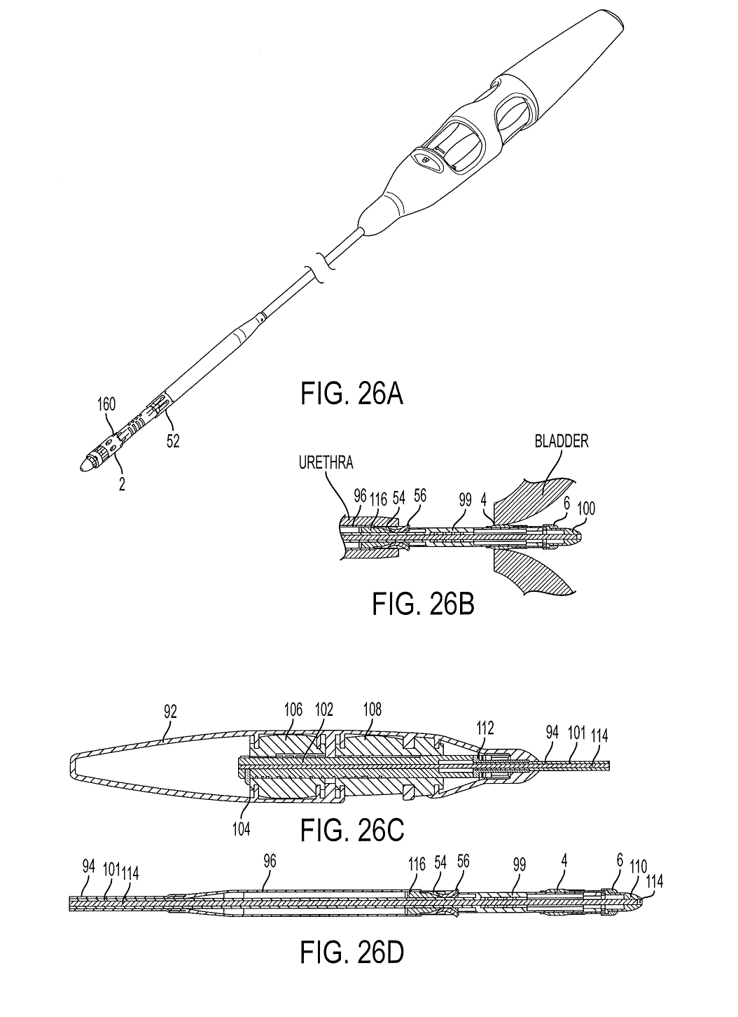

As depicted in FIG. 25, the anastomosis system 1 is inserted through the urethra to position first ring assembly 2 within a first hollow body part, such as a bladder neck, by pushing hollow grip member 103 of handle assembly 92 (not shown) to advance the insertion instrument 90 through the second hollow body part, such as a urethra. FIGS. 26A-26D show the arrangement of the insertion instrument 90 during insertion. As shown, the second ring assembly 52 and first ring assembly 2 are mounted on the second ring assembly mounting portion 162 and first ring mounting portions 160, respectively. Both the first and second ring assemblies 2, 52 are in the undeployed or retracted position. The insertion instrument 90 is in the “Locked” position.

Turning now to FIGS. 27A-27D, the deployment of the first ring securement elements 20 of the first ring assembly 2 is shown. As shown in Fig, 27B, once the first ring assembly 2 is aligned at a suitable position within the first hollow body part (e.g. bladder neck), the rotary selection knob 108 is rotated counter-clockwise (in the depicted embodiment, the angle of rotation is 72°; however, other degrees of rotation are contemplated). As discussed above with respect to FIG. 14B, rotation of the rotary selection knob 108 from the initial “Locked” position selects a deployment position, such as the “Bladder” deployment position shown here. When the rotary selection knob 108 is in the “Bladder” deployment position, counter-clockwise rotation of the rotary actuation knob 106 with respect to the handle assembly 92 results in axial translation of the actuation shaft 102 in the direction of the handle assembly, i.e. in the proximal direction. As shown in FIG. 27C, proximal translation of the actuation shaft 102 also carries the deployer shaft 114 in the proximal direction with respect to the handle assembly 92 and through the lumens 117 and 118 of the flexible tube 94 and implant support 98 (indicated by arrows “x” in FIGS. 27A, 27C and 27D).

As shown in FIG. 27D, because the deployer 100 is mounted on the deployer shaft 114 and the first central ring 6 is mounted to the deployer 100, translation of the deployer shaft 114 towards the handle 92 carries the first central ring 6 axially towards the first collar 4. As discussed with respect to FIGS. 4 and 5 and shown in FIG. 27B, as the first central ring 6 advances into the first collar 4, the guide surfaces 34 of the first collar 4 displace the first ring securement elements 20, which are urged to bend and deploy radially outward and proximally from the first collar 4 (shown by arrow “y” in FIGS. 27A, 27C and 27D).

As shown in FIG. 27B, the deployment of the first ring securement elements 20 when the first ring assembly 2 is in position in the first hollow body part (e.g., bladder neck) causes the first ring securement elements 20 to pierce and engage the hollow body part tissue. As shown, the first ring securement elements 20 secure the first hollow body part (e.g., bladder neck) by being driven into the tissue in a generally proximal and radially outward direction. Although not shown, a surgeon may also compress the first hollow body part (e.g., bladder neck) tissue around the first ring assembly 2 to ensure that the first ring securement elements 20 securely engage the first hollow body part. Additionally or alternatively, the surgeon may gently pull the insertion instrument 90 in the proximal direction with respect to the first hollow body part (e.g., bladder) to secure and/or maintain engagement of the first ring securement elements 20 with the first hollow body part.

As shown in FIG. 27A, the first ring assembly 2 can be undeployed by clockwise rotation of the rotary actuation knob 106 with respect to the hollow grip member 103 to cause the deployer shaft 114 and deployer 100 to axially extend in the distal direction with respect to the first collar 4, thereby carrying the first central ring 6 away from the first collar 4. As a result, if proper attachment to the first hollow body part (e.g., bladder) is not achieved initially, the first ring securement elements 20 may be retracted and redeployed.

Turning now to FIGS. 28A-28D, partial deployment of the second ring assembly 52 to engage the second hollow body part (e.g. urethra) is shown. As shown in FIGS. 28A and B, once the first ring assembly 2 is secured in the first hollow body part (e.g., bladder), the second ring assembly 52 is aligned at a suitable position within the second hollow body part (e.g., urethra neck), the rotary selection knob 108 is rotated counter-clockwise (in the depicted embodiment, the angle of rotation is 72°; however, other degrees of rotation are contemplated). As discussed above with respect to FIG. 14C, rotation of the rotary selection knob 108 from the “Bladder” deployment position selects the “Urethra” deployment position.

When the rotary selection knob 108 is in the “Urethra” deployment position, counter-clockwise rotation of the rotary actuation knob 106 with respect to the handle assembly 92 results in axial translation of the actuation shaft 102 in the proximal direction with respect to the hollow grip member 103. Furthermore, because the adapter 112 is engaged by the actuation shaft 102 (as seen in FIG. 14C) when the rotary selection knob 108 is in the “Urethra” deployment position, proximal translation of the actuation shaft 102 carries the adapter 112 and the implant support 98 mounted thereto in a coordinating proximal movement. Thus, as shown in FIG. 28C, proximal translation of the actuation shaft 102 and adapter 112 when the rotary selection knob 108 is in the “Urethra” deployment position carries the implant support 98 and deployer shaft 114 in the proximal direction through the lumens 117 and 119 of the flexible tube 94 and urethra side cam 116 and the implant mounting portion 99 of the implant support 98 into the outer housing 96 (indicated by arrows “x” in FIGS. 28A, 28B, 28C and 28D).

As shown in FIG. 28D, the second collar 54 is mounted on the second collar mounting portion 97 of the outer housing 96, such that proximal translation of the implant mounting portion 99 of the implant support 98 through the outer housing 96 carries the second central ring 56 into sliding engagement with the second collar 54. Thus, the proximal translation of the implant support 98 drives the second ring securement elements 62 into contact with the angled second ring securement element engagement surface 82 of the second collar 54 and the urethra side cam 116. As discussed with respect to FIG. 6 and shown in FIG. 28B, engagement of the second ring securement elements 62 with the angled second ring securement element engagement surfaces 82 of the second collar 54 and the urethra side cam 116 displaces the second ring securement elements 62, thereby urging the second ring securement elements 62 radially outward (shown by arrow “y” in FIGS. 28A, 28B and 28D). The second central ring 56 slides into the second collar 54 until the second ring securement element mounting member 64 of the second central ring 56 contacts the proximal ring base 76 of the second collar 54.

As shown in FIG. 28B, the radial deployment of the second ring securement elements 62 when the second ring assembly 52 is in position in the second hollow body part causes the second ring securement elements 62 to pierce and engage the second hollow body part, such as a urethra neck. As shown, the second ring securement elements 62 secure the second hollow body part by being driven into the tissue in a generally radially outward direction.

Additionally, as shown in FIG. 28A, the second ring assembly 52 can also be undeployed by clockwise rotation of the rotary actuation knob 106 with respect to the hollow grip member 103 to cause the implant support 98 and deployer 100 to axially extend in the distal direction with respect to the second collar 54, thereby carrying the second central ring 56 away from the second collar 54.

Turning now to FIGS. 29A-29D, full deployment and securement of the second ring securement elements 62 in the second hollow body part (i.e., urethra) is shown. As shown in FIG. 29B, once the first ring assembly 2 is secured in the first hollow body part (e.g., bladder) and the second ring assembly 52 is partially deployed within the second hollow body part (i.e., urethra neck), the rotary selection knob 108 is rotated counter-clockwise (in the depicted embodiment, the angle of rotation is 72°; however, other degrees of rotation are contemplated). As discussed above with respect to Fig, 14D, rotation of the rotary selection knob 108 from the “Urethra” deployment position selects the “Anastomosis” deployment position.

When the rotary selection knob 108 is in the “Anastomosis” deployment position, counter-clockwise rotation of the rotary actuation knob 106 with respect to the hollow grip member 103 results in axial translation of the actuation shaft 102 in the proximal direction with respect to the hollow grip member 103. As shown in FIG. 29C, proximal translation of the actuation shaft 102 when the rotary selection knob 108 is in the “Anastomosis” deployment position carries the implant support shaft 101 and deployer shaft 114 further in the proximal direction through the lumens 117 and 119 of the flexible tube 94 and urethra side cam 116 and the implant mounting portion 99 of the implant support 98 further into the outer housing 96 (indicated by arrows “x” in FIGS. 29A, 29B and 29D).

As shown in FIG. 29B, in the “Anastomosis” deployment position, the second ring securement element mounting member 64 engages the second collar 54, thereby preventing further sliding of the second central ring 56 into the second collar 54. Furthermore, because the second collar 54 is mounted on the outer housing 96 as shown, the outer housing 96 causes the second collar 54 and second central ring 56 to resist further axial movement. Thus, with the second ring assembly 52 resisting further axial translation with respect to the outer housing 96, the force applied by proximal translation of implant support 98 with respect to the outer housing 96 drives the second ring support members 168 (see FIG. 13A) inward, thereby disengaging the second central ring 56 from the implant support 98.

With the second central ring 56 disengaged from the implant support 98, the implant mounting portion 98 can translate proximally with respect to the second central ring 56 when the implant support 98 is carried proximally by the actuation shaft 102. Furthermore, as the implant mounting portion 98 translates proximally with respect to the second central ring 56, the second ring securement element engaging cam members 163 of the implant mounting portion 99 of the implant support 98 are driven into contact with the second ring securement element cam surfaces 72 of the second ring securement elements 62, which are pivoted to extend into the lumen 60 of the second central ring 56. Engagement of the second ring securement element engaging cam members 163 with the second ring securement element cam surfaces 72 of the second ring securement elements 62 during proximal translation of the implant support 98 urges the second ring securement elements 62 to pivot further outward until the second ring securement element cam surfaces 72 are axially aligned with the second ring securement element mounting members 64. As shown in Fig. 29B, the second ring securement elements 62 are fully deployed and are generally directed distally to secure the second hollow body part, such as a urethra.

Turning now to FIGS. 30A-30D, approximation of the first ring assembly 2 and the second ring assembly 52 and anastomosis of the hollow body parts, such as a urethra and bladder, is shown. As shown in FIG. 30B, the first ring assembly 2 is secured in the bladder and the second ring assembly 52 is fully deployed and secured within the urethra neck. The rotary selection knob 108 is not rotated and the insertion instrument 90 remains in the “Anastomosis” deployment position. Counter-clockwise rotation of the rotary actuation knob 106 with respect to the hollow grip member 103 results in axial translation of the actuation shaft 102 in the proximal direction with respect to the hollow grip member 103. As shown in FIG. 30C, further proximal translation of the actuation shaft 102 when the rotary selection knob 108 is in the “Anastomosis” deployment position following full deployment of the second ring assembly 52 carries the implant support shaft 101 and deployer shaft 114 further in the proximal direction with respect to the flexible tube 94, urethra side cam 116 and outer housing 96 and through the lumens 117 and 119 of the flexible tube 94 and urethra side cam 116 (indicated by arrows “x” in FIGS. 29A to 29D).

Furthermore, as shown in FIG. 30D, the implant mounting portion 99 of the implant support 98 is also carried further into the outer housing 96, though lumens 60 and 80 of the second central ring 56 and second collar 54 and into engagement with the urethra side cam 116. Proximal movement of the implant mounting portion 99 of the implant support 98 through the outer housing 96 displaces the urethra side cam 116 and the urethra side cam 116 is pushed proximally with respect to the outer housing 96 by the implant support 98. Additionally, proximal translation of the implant mounting portion 99 of the implant support 98 carries the first ring assembly 2, and the first hollow body part tissue (i.e., bladder tissue) secured thereto towards contact with the second ring assembly 52, and the second hollow body part tissue (i.e., urethra tissue) secured thereto. As shown, the cut portion of bladder B1 at least partially engages the cut portion of the urethra U1 to form an end-to-end anastomosis, although end-to-end anastomosis of other hollow body parts may be achieved by the same or similar methods.

As shown in FIG. 30D, when the first ring assembly 2 is brought into engagement with the second ring assembly 52, the first ring assembly 2 and second ring assembly 52 couple together due to engagement of the first ring interconnecting elements 47 with the second ring interconnecting elements 84. Specifically, due to axial alignment of the first ring interconnecting elements 47 with the second ring interconnecting elements 84, translation of the first ring assembly 2 into contact with the second ring assembly 52 urges the first ring interconnecting elements 47 into connecting engagement with the second ring interconnecting elements 84 by a snap- or press-fit connection.

Furthermore, due to the axial alignment of the second central ring locks 86 with the support surfaces 50 of the first collar 4, translation of the first ring assembly 2 into engagement with the second ring assembly 52 urges the second central ring locks 86 against the support surfaces 50 of the first collar 4 and inwardly displaces the first collar locking member 166. Inward displacement of the first collar locking member 166 disengages the first collar locking member 166 from the first collar 4 and allows the implant mounting portion 99 of the implant support 98 to slide through lumens 60 and 80 of the second collar 54 and the second central ring 56.

Simultaneously, due to the axial alignment of the second ring securement element locking members 48 of the first collar 4 and the second ring securement element cam surfaces 72, translation of the first ring assembly 2 into engagement with the second ring assembly 52 urges the second ring securement element locking members 48 of the first collar 4 into engagement with the second ring securement element cam surfaces 72. Engagement of the second ring securement element locking members 48 of the first collar 4 with the second ring securement element cam surfaces 72 resists pivoting of the second ring securement element cam surfaces 72 into the lumen 60 of the second central ring 56 and supports the second ring securement elements 62 in the fully deployed position.

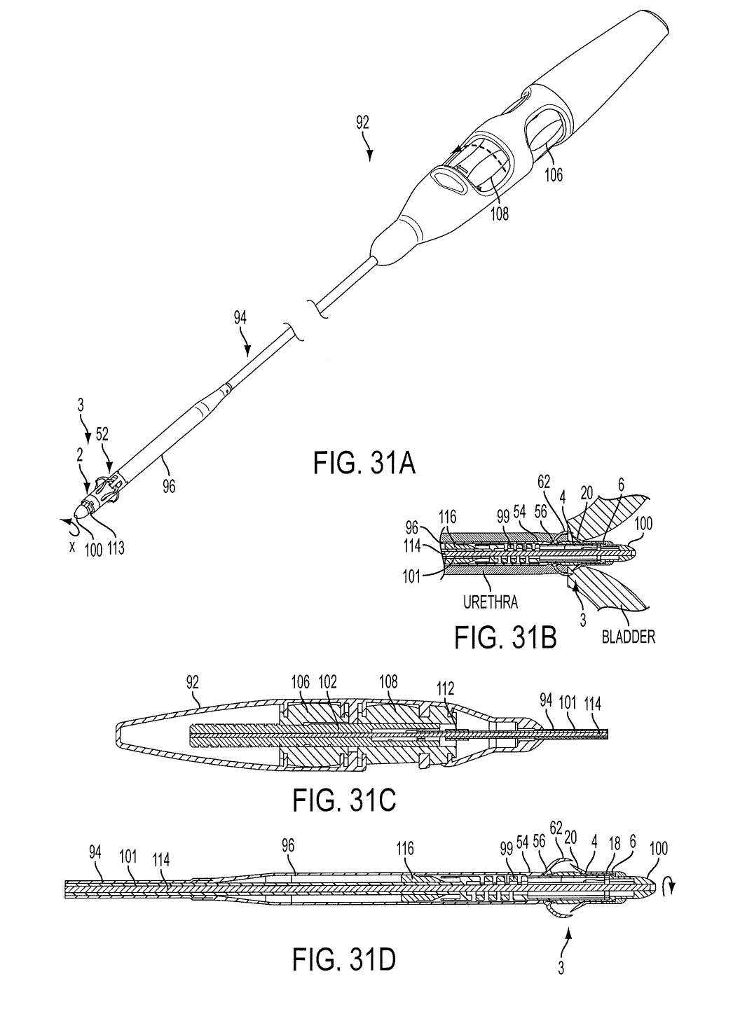

Turning now to FIGS. 31A-31D, release of the first ring assembly 2 and second ring assembly 52 from the insertion instrument 90 following coupling of the first and second ring assemblies 2, 52 to form an anastomosis is shown. As shown in FIG. 31B, once the first ring assembly 2 and second ring assembly 52 are secured to the tissue and coupled together, the rotary selection knob 108 is rotated counter-clockwise by 72°. Rotation of the rotary selection knob 108 from the “Anastomosis” deployment position selects the “Release” position.

Rotation of the rotary selection knob 108 to the “Release” position rotates the actuation shaft 102 counter-clockwise with respect to the hollow grip member 103. Rotation of the actuation shaft 102 causes circumferentially extending recess 134 of the actuation shaft 102 to slide against the hollow grip release detent 133 of the hollow grip member 103 and the actuation shaft detent 136 to engage the hollow grip release detent 133. Furthermore, because the deployer shaft 114 is mounted to the actuation shaft 102 and the deployer 100 is mounted to the deployer shaft 114, the deployer 100 also rotates counter-clockwise with respect to the hollow grip member 103 of the handle assembly 92.

As seen in FIG. 31A, rotation of the deployer 100 causes the deployer detent 113 of the deployer 100 to slide through circumferentially extending deployment slot 18 of the first central ring 6 and into device release groove 16. When the deployer detent 113 of the deployer 100 is positioned in the device release groove 16 of the first central ring 6, the deployer 100 can slide through the lumen 10 of the first central ring 6. Furthermore, in the “Release” position, the deployer 100 and implant mounting portion 99 of the implant support 98 can slide through the lumens 35, 60, 80 of the first collar 4, second central ring 56 and second collar 54.

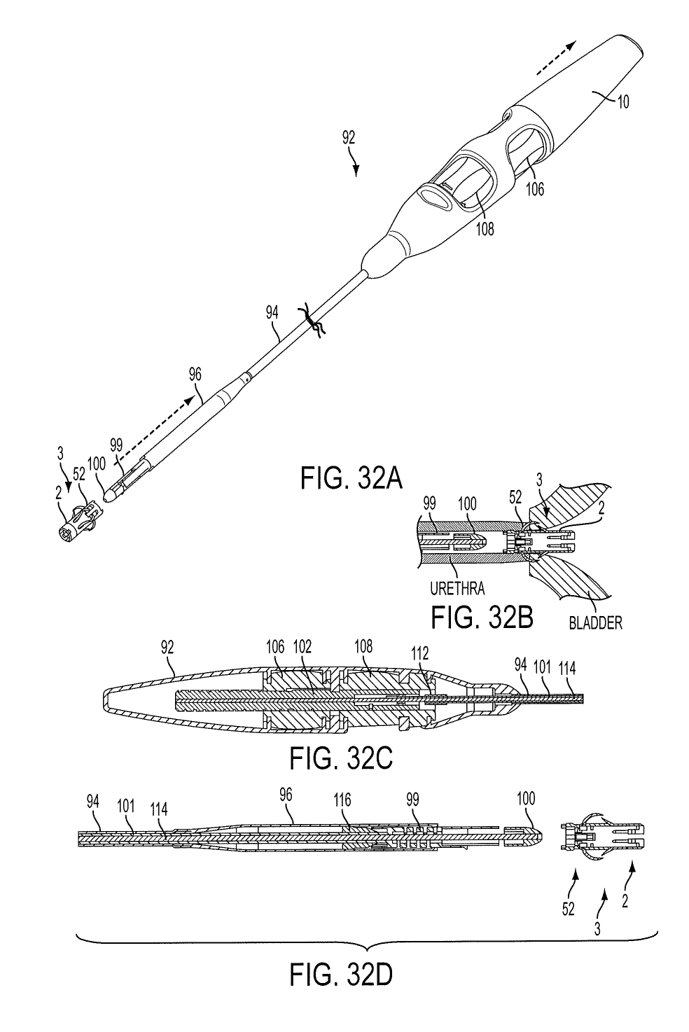

Turning now to FIGS. 32A-32D, the withdrawal of the insertion instrument 90 from the body following release of the first and second ring assemblies 2, 52 is shown. As shown in FIGS. 32B, the second and first ring assemblies 52, 2 are secured to the tissue. Accordingly, as shown in FIG. 32A, proximal translation of the handle assembly 92 through the second hollow body part withdraws the insertion instrument 90 from the patient. The instrument engaging element 88 releases the second collar 54 from the outer housing 96 of the insertion instrument 90 when the second ring assembly 52 is secured to the second hollow body part and the insertion instrument 90 is translated away from the second ring assembly 52 leaving the second and first ring assemblies 52, 2 coupled together to hold the hollow body parts, such as a urethra and bladder, in anastomosis. The second and first ring assemblies 52, 2 may be removed after a period of healing or, alternatively, may be permitted to biodegrade in place.

The preferred materials for the ring assembly 3 are now discussed. However, it will be understood that this discussion of materials can apply equally to all embodiments disclosed and contemplated herein. The ring assembly 3 is preferably formed of materials that are compatible with the environment (e.g. range of pH, variable constituents of bodily fluids such as urine and variable flow of such fluids). The entirety of the ring assembly 3 may be formed from resorbable material(s) or at least a portion of the assembly may be formed from permanent material(s). Alternatively, one or more portions of the ring assembly 3 may be formed of resorbable material(s) while one or more other portions are formed from permanent material(s). In some embodiments, the first ring and second ring securement elements 20 and 62, in particular, are formed from resorbable material, whereas other portions are formed from permanent materials. In some examples, a ring assembly 3 can be formed with a resorbable element that connects two non-resorbable elements and breaks down to permit the ejection of the permanent elements in the urine stream. In other examples, portions of the ring assembly may be formed from mixtures of different resorbable materials and/or different permanent materials.

As used herein, “permanent materials” refers to materials that are not expected to undergo dramatic changes in strength or composition during the period of time that the ring assembly 3 is needed to allow healing of tissues and the establishment of a tissue-based channel for urine flow. Permanent materials include, but are not limited to, polymeric materials or metals. Examples of permanent polymeric materials include PEEK (polyether ether ketone), polyethylene, polypropylene and others currently used in medical devices both in the United States and worldwide. Permanent metals include those used in surgery such as, but not limited to, stainless steel and titanium, both in a range of compositions and alloys.

As used herein, “resorbable materials” refers to materials that exhibit the ability to change over time, such as breaking down and eventually being eliminated from the body of the patient. Resorbable materials include, but are not limited to, bioabsorbable and biodegradable materials. Preferably, resorbable materials may be used as elements of implantable devices where over a period of time the implant breaks up and is absorbed, shed, or ejected from the body.

Resorbable materials are well known in the literature. See, Principles of Tissue Engineering (Lanza and Vacanti, eds., Elsevier Academic Press 3d ed. 2007) (1997), incorporated herein by reference in its entirety. Suitable resorbable materials include, but are not limited to, homopolymers and co-polymer blends from families including polylactic acid, polyglcolic acid, s-caprolactone, and trimethylene carbonate. Other resorbable polymers may include polyphosphazenes, polydioxanones, polyanhydrides and polyurethane materials. Additionally, materials based on naturally occurring substances including, but not limited to polyhydroxyalkanoates, chitin and its derivatives, cellulose and certain other starches that can be fabricated to useful forms may be used. Additionally, suitable resorbable materials may comprise metals, such as magnesium, that can be broken down by the body when used as an implantable device. In one embodiment of the device, representative resorbable materials may comprise blends of 10:90 and 50:50 (both polyglycolide:polylactide blends), which are materials with degradation times that vary from 1-3 months. Alternatively, representative resorbable materials may comprise blends of 82:18 or 85:15 (both polyglycolide:polylactide blends), which are materials with degradation times that vary from 6-12 months. Material degradation times may be altered by changing processing methods (including exposure to heat and/or moisture during or after processing) as well as sterilization method. Also, environmental characteristics, such as pH and temperature, will also affect implant characteristics, such as degradation time.

Additionally, the ring assembly may be formed from ceramics, such as calcium phosphate and hydroxyapatite based ceramics. By way of background, see e.g., Biomaterials Science: An Introduction to Materials Medicine 64-73 (Buddy D. Ratner ed., Academic Press, Ltd., 1996), incorporated herein by reference in its entirety. The ceramic materials may be permanent or resorbable depending on their chemistry, blending and even manufacturing methods used. The ring assembly 3 may also be formed of a biocompatible, resorbable and/or permanent materials, such as those described in the following US Patents, the contents of which are incorporated by reference in their entirety herein: U.S. Pat. No. 5,432,395, U.S. Pat. No. 4,976,715, U.S. Pat. No. 5,273,964, U.S. Pat. No. 4,157,378, U.S. Pat. No. 4,429,691, U.S. Pat. No. 4,612,053, U.S. Pat. No. 4,684,673, U.S. Pat. No. 4,737,411, U.S. Pat. No. 4,849,193, U.S. Pat. No. 4,880,610, U.S. Pat. No. 4,917,702, U.S. Pat. No. 4,938,938, U.S. Pat. No. 4,959,104, U.S. Pat. No. 5,034,059, U.S. Pat. No. 5,037,639, U.S. Pat. No. 5,047,031, U.S. Pat. No. 5,053,212, U.S. Pat. No. 5,085,861, U.S. Pat. No. 5,129,905, U.S. Pat. No. 5,149,368, U.S. Pat. No. 5,152,836, U.S. Pat. No. 5,164,187, U.S. Pat. No. 5,178,845, U.S. Pat. No. 5,262,166, U.S. Pat. No. 5,279,831, U.S. Pat. No. 5,281,265, U.S. Pat. No. 5,286,763, U.S. Pat. No. 5,336,264, U.S. Pat. No. 5,427,754, U.S. Pat. No. 5,470,803, U.S. Pat. No. 5,496,399, U.S. Pat. No. 5,516,532, U.S. Pat. No. 5,522,893, U.S. Pat. No. 5,525,148, U.S. Pat. No. 5,542,973, U.S. Pat. No. 5,545,254, U.S. Pat. No. 5,562,895, U.S. Pat. No. 5,565,502, U.S. Pat. No. 5,605,713, U.S. Pat. No. 5,650,176, U.S. Pat. No. 5,665,120, U.S. Pat. No. 5,691,397, U.S. Pat. No. 5,700,289, U.S. Pat. No. 5,782,971, U.S. Pat. No. 5,846,312, U.S. RE33,161, U.S. RE33,221, U.S. Pat. No. 5,658,593, U.S. Pat. No. 6,752,938, U.S. Pat. No. 8,048,443, and U.S. Pat. No. 8,048,857.

In all of the disclosed ring assembly embodiments disclosed herein, a sealant can be included between the first ring assembly and the second ring assembly for sealing the ring assemblies together. Any such sealant can be moisture activated. Moreover, the sealant may be a 2-part product that only activates when the two parts are in contact similar to a 2-part epoxy. Thus, if a 2-part sealant is used, one part can be included on the first ring assembly and the other part can be included on the second ring assembly such that when the first and second ring assemblies are coupled together, the two parts will contact each other and activate thereby sealing the assemblies together.

As mentioned above, when the ring assembly 3 is formed from resorbable and/or biodegradable materials, it gradually degrades after implantation in the body. Preferably, the material is selected to degrade at a slower rate than the natural healing process, so as to allow healing of the anastomosis before degradation. For example, the ring assembly 3 can be formed from a material that will (i) remain intact for approximately six weeks after implantation before degradation and (ii) be completely resorbed or degraded after twelve weeks. Thus, the ring assembly 3 can be removed or expelled from the patient's body without a follow-up surgical procedure when the ring assembly 3 is no longer needed to hold the anastomosis. In the interim, the ring assembly 3 permits bodily fluids, such as urine, to flow from the first hollow body part, such as a bladder, through the lumens (10, 35 60, and 80) of the first and second ring assemblies 2, 52 and into the second hollow body part (e.g., urethra) while the anastomosis is healing. Preferably, the ring assembly 3 forms a leak-proof passageway, so as to reduce or eliminate the chance of leakage of urine into the abdominal cavity. The flow of bodily fluid, such as urine, through the ring assembly may operate to degrade the ring assembly and carry non-resorbed materials and portions of the ring assembly out of the body.

It will be noted that in some other embodiments, the mating screw threads can be reversed so that the operations described are performed by rotating the components in the opposite angular directions. In some other embodiments, the ring-mounting steps and the securement element-deploying steps can be performed by other components of the system. In some other embodiments, the securement elements can be spring-biased to their deployed positions and deployed by actuation of a release member.

It should be understood that, although this disclosure describes different embodiments separately, that one skilled in the art may combine the features of different embodiments without departing from the anastomosis devices and system disclosed herein. For example, one skilled in the art may incorporate the securement elements and deployment mechanism of one embodiment in a first ring assembly (e.g., rigid pivotable hooks, etc.) while incorporating a different securement element and deployment mechanism (e.g., resilient flexible hooks, etc.) in the second ring assembly. Furthermore, it should be apparent to those skilled in the art that the tissue capture elements referred to as “upper” and “lower” may be adapted for use interchangeably. In other words, a first ring shown engaging the bladder or described as “upper” may be adapted to engage the urethra or used as a “lower” ring. Likewise, a second ring shown engaging the urethra or described as “lower” may be adapted to engage the bladder or used as an “upper” ring.

It should also be understood that although the present disclosure may describe deployment or actuation of certain structures by moving or translating a component or structure distally or proximally with respect to another component or structure, those skilled in the art will understand that deployment of the same structures may be accomplished by moving or translating such components in a different manner. For example, while the present disclosure may describe deploying securement elements by moving a central ring proximally towards an upper collar, deploying the same securement elements may be achieved by moving the upper collar distally towards the central ring. Moreover, it should be understood that although the present disclosure describes deployment of certain structures as occurring when one component is moved towards another component that is held stationary, those skilled in the art will understand that the deployment of such structures, may be accomplished by moving both components towards each other.

Additionally, all US patents, applications, and published literature cited herein are incorporated by reference in their entireties.

It is to be understood that this invention is not limited to the specific devices, methods, conditions, or parameters described and/or shown herein, and that the terminology used herein is for the purpose of describing particular embodiments by way of example only. Thus, the terminology is intended to be broadly construed and is not intended to be limiting of the disclosed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein. And any dimensions shown in the attached drawings are representative and not limiting of the invention, as larger or smaller dimensions can be used as desired.

Although the present invention has been described above in terms of exemplary embodiments, it is not limited thereto. Rather, the appended claims should be construed broadly to include other variants and embodiments of the invention which may be made by those skilled in the art without departing from the scope and range of equivalents of the invention.

1-13. (canceled)

14. An anastomosis device comprising:
a first ring assembly comprising:
a first ring having:
a cylindrical wall defining a lumen therethrough and including a first end and a second end;
a plurality of guide structures in the cylindrical wall; and
a plurality of mounting elements extending from the second end; and
a first central ring having (i) a cylindrical wall defining a lumen therethrough and including a first end and a second end, and (ii) a plurality of first securement elements extending from the first end; and
a plurality of first connecting elements; and
a second ring assembly comprising:
a second ring having (i) a cylindrical wall defining a lumen therethrough and including a first end and a second end, and (ii) a plurality of axial slots in the cylindrical wall;
a second central ring having (i) a cylindrical wall defining a lumen therethrough and including a first end and a second end, and (ii) a plurality of second securement elements extending from the first end; and
a plurality of second connecting elements.

15. The anastomosis device of claim 14, wherein the first central ring is movable from a first position to a second position with respect to the first ring, and wherein the second position is closer to the second ring assembly than the first position.

16. The anastomosis device of claim 14, wherein, when the first central ring is in the first position, the plurality of first securement elements are in an undeployed position.

17. The anastomosis device of claim 16, wherein the cylindrical wall of the first ring comprises an outer circumference, and wherein, when the first central ring is in the first position, no portion of the plurality of first securement elements extends past the outer circumference of the cylindrical wall.

18. The anastomosis device of claim 17, wherein, when the first central ring is in the first position, at least a portion of the plurality of first securement extends at least partially into the guide structures of the cylindrical wall.

19. The anastomosis device of claim 14, wherein the cylindrical wall of the first ring comprises an outer circumference, and wherein, when the first central ring is in the second position, at least a portion of the plurality of first securement elements extends radially outward through the guide structures in the cylindrical wall of the first ring, past the outer circumference of the cylindrical wall.

20. The anastomosis device of claim 14, wherein the plurality of second securement elements are movably connected to the second central ring.

21. The anastomosis device of claim 14, the second ring comprising an outer circumference,
wherein the second central ring is movable from a first position to a second position with respect to the second ring,
wherein, when the second central ring is in the first position, the second securement elements are disposed generally within the plurality of axial slots in the cylindrical wall, and
wherein, when the second central ring is in the second position, the second securement elements extend at least partially through the plurality of axial slots radially outward past the outer circumference of the second central ring.

22. The anastomosis device of claim 14, wherein the plurality of second connecting elements are adapted to matingly engage the plurality of first connecting elements to couple the first ring assembly and the second ring assembly together.

23. The anastomosis device of claim 22, wherein at least one of the first connecting elements and at least one of the second connecting elements includes at least one ratcheting element capable of providing a variable coupling distance between the first ring assembly and the second ring assembly.

24. The anastomosis device of claim 23, wherein the ratcheting element is selected from the group consisting of notches, teeth and detents.

25. The anastomosis device of claim 22, wherein, when the plurality of second connecting elements of the second ring engage the plurality of first connecting elements of the first ring, the lumen of the first ring and the lumen of the second ring are generally coaxial, thereby forming a single lumen extending through the anastomosis device.

26. The anastomosis device of claim 14, wherein the first ring assembly is adapted for insertion into a bladder neck and the second ring assembly is adapted for insertion into a urethra.

27. The anastomosis device of claim 14, wherein the first and second ring assemblies are at least partially formed from a biodegradable material.

28-54. (canceled)
